(19) United States
(12) Patent Application Publication
(10) Pub. No.: US 2025/0101522 A1
(43) Pub. Date: Mar. 27, 2025

(54) BRCA1 PROMOTER METHYLATION IN SPORADIC BREAST CANCER PATIENTS DETECTED BY LIQUID BIOPSY

(71) Applicant: GUARDANT HEALTH, INC., Palo Alto, CA (US)

(72) Inventors: Jennifer YEN, San Mateo, CA (US); Sai CHEN, San Diego, CA (US)

(21) Appl. No.: 18/634,561

(22) Filed: Apr. 12, 2024

Related U.S. Application Data
(60) Provisional application No. 63/509,917, filed on Jun. 23, 2023.
(60) Provisional application No. 63/495,688, filed on Apr. 12, 2023.

(51) Int. Cl. C12Q 1/6886

(52) U.S. Cl. CPC C12Q 1/6886; C12Q 2600/106; C12Q 2600/154

(57) ABSTRACT

Described herein are methods such as diagnoses to select therapies for personalized cancer treatment by simultaneously detecting genomic and epigenomic attributes from a single patient sample, including quantifying promoter methylation and applications for ascertaining methylation patterns associated with epigenetic allelic status.

### CROSS-REFERENCE TO RELATED APPLICATIONS

This application claims the benefit of U.S. provisional patent application No. 63/509,917 filed Jun. 23, 2023, and 63/495,688 filed Apr. 12, 2023, which are each incorporated by reference herein in its entirety.

### FIELD OF THE INVENTION

Described herein are methods such as diagnoses to select therapies for personalized cancer treatment by simultaneously detecting genomic and epigenomic attributes from a single patient sample, including quantifying promoter methylation.

### BACKGROUND

Therapy selection for cancer patients is imprecise. DNA, RNA and protein from patient samples are often analyzed for patterns that can predict response to particular treatments. These biomarkers can range from single genes (e.g. EGFR, via real-time PCR) and proteins (e.g. HER2, via immuno-histochemistry) to complex genomic signatures (e.g. Tumor Mutational Burden, via Next-Generation Sequencing). Testing workflows for multiple types of analytes are generally separate and cannot be combined due to incompatibility in separation, chemical, and quantification processes etc. As a result, diagnostic tests do not examine the whole range of informative biomarkers and multiple, separate tests must be performed if multiomic results are desired. In most cases multiple tests are not performed and clinical decisions are made based on incomplete information for multiple reasons including lack of sufficient patient samples.

There is a great need in the art for improvements in personalized medicine. By simultaneously interrogating the genomic and epigenomic status of the patient sample, more accurate prediction of therapy effectiveness can be accomplished.

Described herein is simultaneous testing for and incorporating information derived from both the genomic and epigenomic components of the patient sample, such a diagnostic test will be able to take into consideration additional information not available otherwise. Of interest is methylation status, particularly of promoter region, including methylation patterns associated with epigenetic allelic status, which may account for instances genomic alterations fail to account varying efficacy of treatments in patients including for example, parp inhibitors (PARPi).

### SUMMARY OF THE INVENTION

Described herein is a method, comprising: detecting methylation in one or more promoter regions of at least one of a plurality of genes; and generating a plurality of methylation calls to quantify methylation of the one or more promoter regions. In other embodiments, the method includes obtaining a sample. In other embodiments, the method includes having obtained a sample. In other embodiments, the method includes processing the quantities of methylation of the one or more promoter regions to characterize a sample. In other embodiments, the method includes characterizing the sample includes HRD, cancer derived promoter methylation, familial forms of colorectal cancer, or Lynch syndrome tumor types. In other embodiments, the promoter includes a region of 5kb upstream of the transcription start site (TSS), wherein the 5kb region is further refined using one or more of: costume panel regions, methylation peaks found in clinical samples, and excluding peaks found in normal samples. In other embodiments, the TSS is defined at the transcript level. In other embodiments, the TSS is defined at the gene level. In other embodiments, the method includes determining the ratio of the number of molecules that overlap a target region normalized by total positive control molecules. In other embodiments, the method includes determining the ratio includes filtering of a molecule based at least on the number of overlapping CpGs. In other embodiments, the method includes quantifying of methylation of the one or more promoter regions is based on the number of methylated CpGs. In other embodiments, the method includes refining the one or more promoter regions based at least on literature annotations, common methylation peak positions, and/or public datasets. In other embodiments, the genes comprise tumor suppressor genes, HRR genes, and IO genes. In other embodiments, the HRR genes comprise at least BRCA1 and BRCA2.

In other embodiments, the method includes comparing to a minimum methylation threshold derived from a population of training samples. In other embodiments, the training samples comprise cancer-free samples. In other embodiments, the minimum methylation threshold for calling includes at least one of: a minimum molecule count of 1-100 and a minimum methylation score per gene is the max of: 95 quantile in normal+8X105 or Median+5*median absolute deviation. In other embodiments, the method includes quantifying methylation of the one or more promoter regions is predictive of therapy response. In other embodiments, the method includes quantifying methylation of the one or more promoter regions is combined with an MSI-H status.

In other embodiments, the therapy includes one or more of an immune checkpoint inhibitor, poly (ADP-ribose) polymerase (PARP) inhibitor, a kinase inhibitor, or an aromatase inhibitor, or a PI3K and mTOR inhibitor. In other embodiments, the immune checkpoint inhibitor is Pembrolizumab. In other embodiments, the method includes poly (ADP-ribose) polymerase (PARP) inhibitor Olaparib or Talazoparib. In other embodiments, the therapy is a combination of a PI3K and mTOR inhibitor and a poly (ADP-ribose) polymerase (PARP) inhibitor. In other embodiments, the PI3K and mTOR inhibitor is Gedatolisib and the poly (ADP-ribose) polymerase (PARP) inhibitor is Talazoparib.

Described herein is a method comprising determining promoter regions of at least one of a plurality of genes, each obtained from a plurality of samples, determining methylation scores for the promoter regions to generate a plurality of methylation calls and/or quantification of promoter methylation, processing the plurality of methylation calls to generate a prediction that a test sample exhibits a genomic state.

Described herein is a method comprising: determining promoter regions of a plurality of genes, each obtained from a plurality of samples; determining methylation scores for the promoter regions to generate a plurality of methylation calls and/or quantification of promoter methylation; processing the plurality of methylation calls to generate a prediction that a test sample exhibits a genomic state. In other embodiments, the genomic state includes HRD, cancer derived promoter methylation, familial forms of colorectal cancer, or Lynch syndrome tumor types. In other embodiments, the promoter includes a region of 5kb upstream of the TSS, wherein the 5kb region is further refined using one or more of: costume panel regions, methylation peaks found in clinical samples, and excluding peaks found in normal samples. In other embodiments, the TSS is defined at the transcript level. In other embodiments, the TSS is defined at the gene level. In other embodiments, the methylation score is determined as the ratio of the number of molecules that overlap a target region normalized by total positive control molecules. In other embodiments, the molecule supporting the methylation score is filtered based at least on the number of overlapping CpGs. In other embodiments, the promoter regions are refined based at least on literature annotations, common methylation peak positions, and/or public datasets. In other embodiments, the genes comprise tumor suppressor genes, HRR genes, and IO genes. In other embodiments, the HRR genes comprise at least BRCA1 and BRCA2. In other embodiments, the calling includes deriving a minimum methylation threshold from a population of training samples. In other embodiments, the training samples comprise cancer-free samples. In other embodiments, the minimum methylation threshold for calling includes:

Minimum molecule count of 1-100 minimum and/or the minimum methylation score per gene is the max of: 95 quantile in normal+8X105 or Median+5*median absolute deviation. In other embodiments, the promoter methylation call combined with an MSI-H status, is predictive of therapy response. In other embodiments, the therapy includes one or more of an immune checkpoint inhibitor, poly (ADP-ribose) polymerase (PARP) inhibitor, a kinase inhibitor, or an aromatase inhibitor, or a PI3K and mTOR inhibitor. In other embodiments, the immune checkpoint inhibitor is Pembrolizumab. In other embodiments, the poly (ADP-ribose) polymerase (PARP) inhibitor Olaparib or Talazoparib. In other embodiments, the therapy is a combination of a PI3K and mTOR inhibitor and a poly (ADP-ribose) polymerase (PARP) inhibitor. In other embodiments, the PI3K and mTOR inhibitor is Gedatolisib and the poly (ADP-ribose) polymerase (PARP) inhibitor is Talazoparib.

Described herein is a method comprising determining promoter regions of BRCA1 and BRCA2, each obtained from a plurality of samples, determining methylation scores for the promoter regions to generate a plurality of methylation calls, processing the plurality of methylation calls to generate a prediction that a patient exhibits biallelic loss of BRCA1 or BRCA2.

Described herein is a method comprising determining promoter regions of BRCA1 and BRCA2, each obtained from a plurality of samples, determining methylation scores for the promoter regions to generate a plurality of methylation calls, processing the plurality of methylation calls to generate a prediction that a patient exhibits biallelic loss of BRCA1 or BRCA2, determining that the patient is a candidate for treatment with a PARPi.

Described herein is a method comprising determining promoter regions of BRCA1 and BRCA2, each obtained from a plurality of samples, determining methylation scores for the promoter regions to generate a plurality of methylation calls, processing the plurality of methylation calls to generate a prediction that a patient exhibits biallelic loss of BRCA1 or BRCA2, determining that the patient is a candidate for treatment with Gedatolisib and Talazoparib. In other embodiments, the methods include, wherein gedatolisib sensitizes advanced TNBC or BRCA1/2 mutant breast cancers to PARP inhibition with talazoparib.

Described herein is a method including determining promoter regions for MLH1, each obtained from a plurality of samples, determining methylation scores for the promoter region to generate a plurality of promoter methylation calls, determining from genomic data that the patient is BRAF V600E positive, wherein detection of the promoter methylation in a BRAF V600E positive patient identifies the patient as one who may be at risk for genetic/familial forms of colorectal cancer or Lynch syndrome-associated tumor types.

Described herein is a method, comprising: obtaining, by a computing system having one or more hardware processors and memory, sequencing reads derived from a sample of a subject, determining one or more classification regions corresponding to a plurality of genes included in the sample; and determine a methylation level of the one or more classification regions by generating a quantitative measure derived from the sequencing reads in the sample of the subject. In other embodiments, the method includes obtaining a sample. In other embodiments, the method includes having obtained a sample. In other embodiments, the method includes processing the methylation level of the one or more classification regions to characterize the sample. In other embodiments, the method includes characterizing the sample comprises determining HRD status, promoter methylation associated with cancer. In other embodiments, the quantitative measure comprises determining the ratio of the number of molecules that overlap a classification region normalized by total positive control molecules, wherein the molecules exhibit a threshold amount of methylated cytosines. In other embodiments, the quantitative measure is compared to a predetermined threshold value to call methylation status of the one or more classification regions. In other embodiments, determining the ratio comprises filtering of a molecule based at least on a threshold amount of methylated cytosines. In other embodiments, determining a methylation level of the one or more classification regions is based on the number of methylated CpGs. In other embodiments, the e classification regions comprise promoter regions. In other embodiments, the one or more classification regions individually correspond to genomic regions in which a methylation rate of cytosines in the genomic regions of nucleic acids derived from cells obtained from subjects in which cancer is present is different from a methylation rate of cytosines in the genomic regions of nucleic acids derived from cells obtained from subjects in which cancer is not present. In other embodiments, the plurality of samples and the additional sample include cell free nucleic acids. In other embodiments, the method includes performing, by the computing system, a training process using the training data to generate the model, wherein the training process includes: determining, by the computing system, one or more additional weights of individual samples included in the training data based on the indication of cancer for the individual samples being within a threshold confidence level. In other embodiments, the indication of cancer for an individual sample is outside of the threshold confidence level and the method comprises: applying, by the computing system, a penalty to a weight of the individual sample during the training process. The method of any preceding claim, comprising: performing, by the computing system and using the one or more machine learning algorithms, one or more first iterations of the training process for the model using a portion of the training data; and generating, by the computing system, first output data for the model based on the one or more first iterations of the training process, the first output data corresponding to one or more first additional indications of cancer being present in first individual subjects of the plurality of subjects, the first individual subjects corresponding to the portion of the training data. In other embodiments, the method includes combining, by the computing system, the first output data and the training data to produce additional training data; performing, by the computing system, one or more second iterations of the training process for the model using a portion of the additional training data; and generating, by the computing system, second output data for the model based on the one or more second iterations of the training process, the second output data indicating one or more second additional indications of cancer being present in second individual subjects of the plurality of subjects, the second individual subjects corresponding to the portion of the additional training data. In other embodiments, the weights for the individual classification regions of the plurality of classification regions are determined based on the first output data and the second output data. In other embodiments, the method includes determining, by the computing system, that a number of indications of cancer being present that were determined during one or more iterations of the training process are at least a threshold value for one or more samples included in the training data; and determining, by the computing system, that modifications to one or more weights of the model are not modified or are modified by a minimal amount. In other embodiments, the method includes determining, by the computing system, that an additional number of indications of cancer being present that were determined during the one or more iterations of the training process are less than the threshold value for one or more additional samples included in the training data; and determining, by the computing system, that modifications to one or more additional weights of the model are modified by more than the minimal amount. In other embodiments, the method includes combining a plurality of nucleic acids derived from at least one of blood or tissue of a subject with a solution including an amount of methyl binding domain (MBD) proteins to produce a nucleic acid-MBD protein solution; and performing a plurality of washes of the nucleic acid-MBD protein solution with a salt solution to produce a number of nucleic acid fractions, individual nucleic acid fractions having a threshold number of methylated cytosines in regions of the plurality of nucleic acids having at least the threshold cytosine-guanine content. In other embodiments, the wash of the plurality of washes is performed with a solution having a concentration of sodium chloride (NaCl) and produces a nucleic acid fraction of the number of nucleic acid fractions having a range of binding strengths to MBD proteins. In other embodiments, the method includes determining that a first nucleic acid fraction is associated with a first partition of a plurality of partitions of nucleic acids, the first partition corresponding to a first range of binding strengths to MBD proteins; attaching a first molecular barcode to nucleic acids of the first nucleic acid fraction, the first molecular barcode being included in a first set of molecular barcodes associated with the first partition; determining that a second nucleic acid fraction is associated with a second partition of the plurality of partitions of nucleic acids, the second partition corresponding to a second range of binding energies to MBD proteins different from the first range of binding strengths to MBD proteins; and attaching a second molecular barcode to nucleic acids of the second nucleic acid fraction, the second molecular barcode being included in a second set of molecular barcodes associated with the second partition.

In other embodiments, the method includes combining at least a portion of the number of nucleic acid fractions with an amount of restriction enzyme that cleaves molecules with one or more unmethylated cytosines to produce at least a portion of the plurality of samples used to produce the sequencing reads, wherein the threshold amount of methylated cytosines corresponds to a minimum frequency of methylated cytosines within a region having at least the threshold cytosine-guanine content.

Described herein is a method, comprising: obtaining, by a computing system having one or more hardware processors and memory, sequencing reads derived from a sample of a subject, determining one or more classification regions corresponding to a plurality of genes included in the sample, determine a methylation level of the one or more classification regions by generating a quantitative measure comprising the ratio of the number of molecules that overlap a classification region normalized by total positive control molecules, wherein the molecules exhibit a threshold amount of methylated cytosines; and comparing the quantitate measure to a predetermined threshold value to call methylation status of the one or more classification regions.

In various embodiments, determination of a quantitative measure can include combining a plurality of nucleic acids derived from at least one of blood or tissue of a subject with a solution including an amount of methyl binding domain (MBD) proteins to produce a nucleic acid-MBD protein solution; and performing a plurality of washes of the nucleic acid-MBD protein solution with a salt solution to produce a number of nucleic acid fractions. In some instances, individual nucleic acid fractions having a threshold number of methylated cytosines in regions of the plurality of nucleic acids having at least the threshold cytosine-guanine content. Thereafter, a wash of the plurality of washes is performed with a solution having a concentration of sodium chloride (NaCl) and produces a nucleic acid fraction of the number of nucleic acid fractions having a range of binding strengths to MBD proteins.

One may determine that a first nucleic acid fraction is associated with a first partition of a plurality of partitions of nucleic acids, the first partition corresponding to a first range of binding strengths to MBD proteins; attach a first molecular barcode to nucleic acids of the first nucleic acid fraction, the first molecular barcode being included in a first set of molecular barcodes associated with the first partition, and subsequently determine that a second nucleic acid fraction is associated with a second partition of the plurality of partitions of nucleic acids, the second partition corresponding to a second range of binding energies to MBD proteins different from the first range of binding strengths to MBD proteins; and thereafter attach a second molecular barcode to nucleic acids of the second nucleic acid fraction, the second molecular barcode being included in a second set of molecular barcodes associated with the second partition.

In some instances, one may combine at least a portion of the number of nucleic acid fractions with an amount of restriction enzyme that cleaves molecules with one or more unmethylated cytosines to produce at least a portion of the plurality of samples used to produce the sequencing reads, wherein the threshold amount of methylated cytosines corresponds to a minimum frequency of methylated cytosines within a region having at least the threshold cytosine-guanine content.

Additionally, one can combine at least a portion of the number of nucleic acid fractions with an amount of a restriction enzyme that cleaves molecules with one or more methylated cytosines to produce at least a portion of the plurality of samples used to produce the sequencing reads, wherein the threshold amount of unmethylated cytosines corresponds to a maximum frequency of methylated cytosines that are not cleaved within a region having at least the threshold cytosine-guanine content.

### BRIEF DESCRIPTION OF FIGURES

FIG. 1. BRCA1 promoter region. The 11 CpG sites (circles) with core promoter activity shown to be hypermethylated in breast cancer (pink circles), are covered in panel BRCA1 promoter definition. The numbers refer to the nucleotide positions relative to the transcription start for BRCA1.

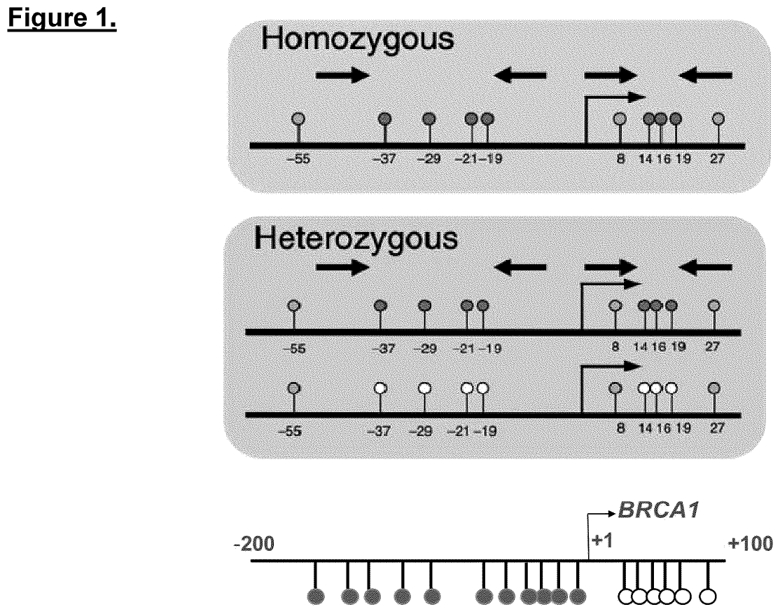

FIG. 2: The 95% Limit of Detection (LoD) for BRCA1. BRCA1 LoD promoter methylation is 0.6%, as determined by titrations of HCC-38, a well characterized breast cancer cell line. Previously, HCC-38 was confirmed to be epigenetically silenced at the BRCA1 locus through promoter methylation, by bisulfite sequencing and RT-PCR (Stefansson 2012, Xu 2010). By comparison, our method did not detect any BRCA1 promoter methylation in the 80 cancer-free donors tested, demonstrating a specificity of 100%

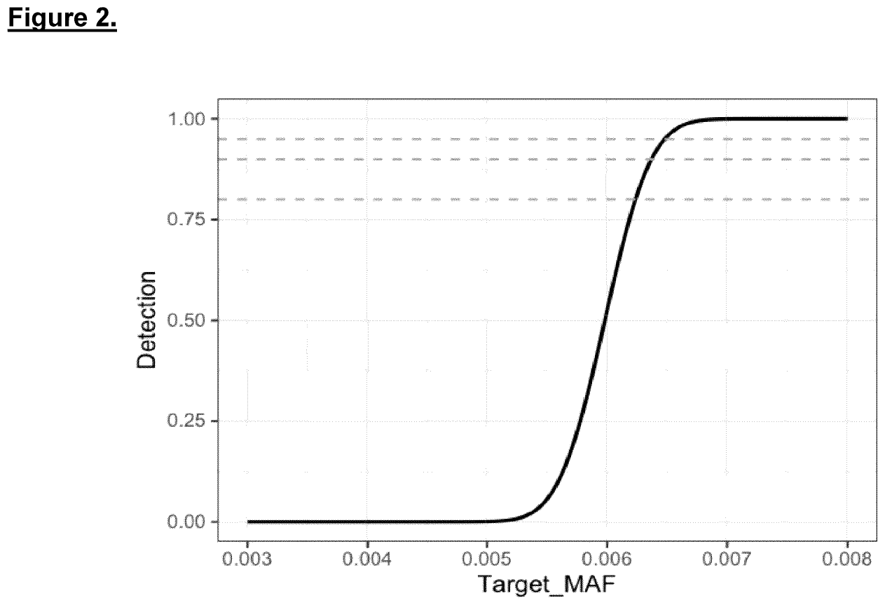

FIG. 3: Prevalence of BRCA1 promoter methylation across cancer types in select patient cohorts. Note that differences in methylation frequencies may be attributed to the unselected, non-random patient subtype composition in the GuardantInfinity cohort, as well stage of cancer (wherein patients may have lost methylation over the course of treatment), and may not be directly comparable to patient cohorts in TCGA. Abbrev: Ovarian (OVCA), Breast (BRCA), Bladder (BLCA), Lung Adenocarcinoma (LUAD), Colorectal Adenocarcinoma (COAD), Lung Squamous Cell Carcinoma (LUSC), Melanoma (SKCM).

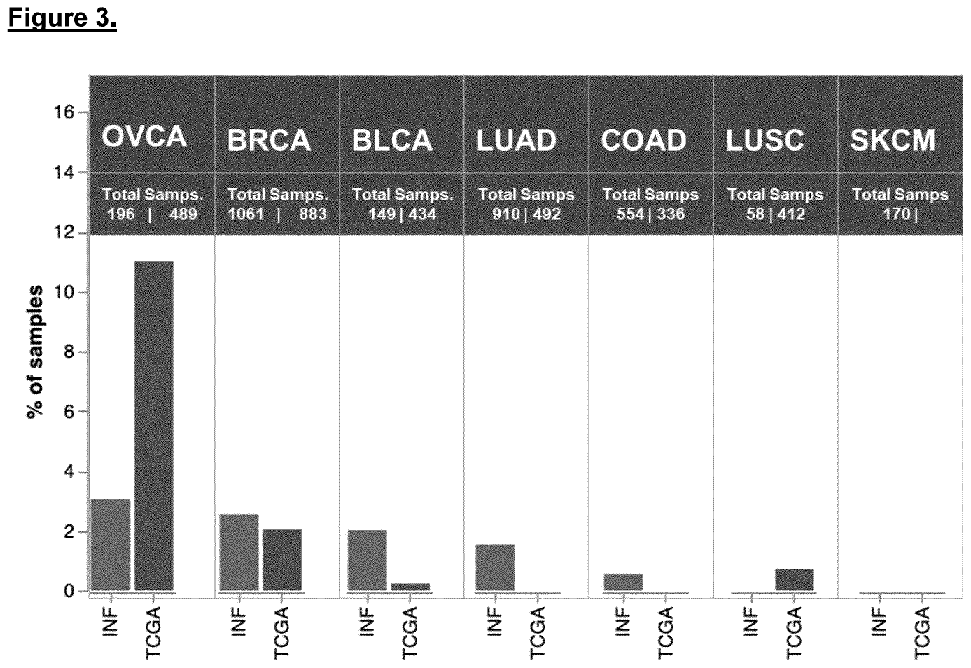

FIG. 4: Oncoprint analysis of epigenetic and genomic alterations in HRR genes. Pathogenic was defined as any nonsense, frameshift, rearrangement or pathogenic ClinVar missense mutations in the HRR genes above. Somatic truncating mutations in ATM and CHEK2 were omitted from this analysis due to possible interference from clonal hematopoiesis. Promoter methylation is highlighted in pink—note that these alterations is majority mutually exclusive with other pathogenic alterations in other HRR genes.

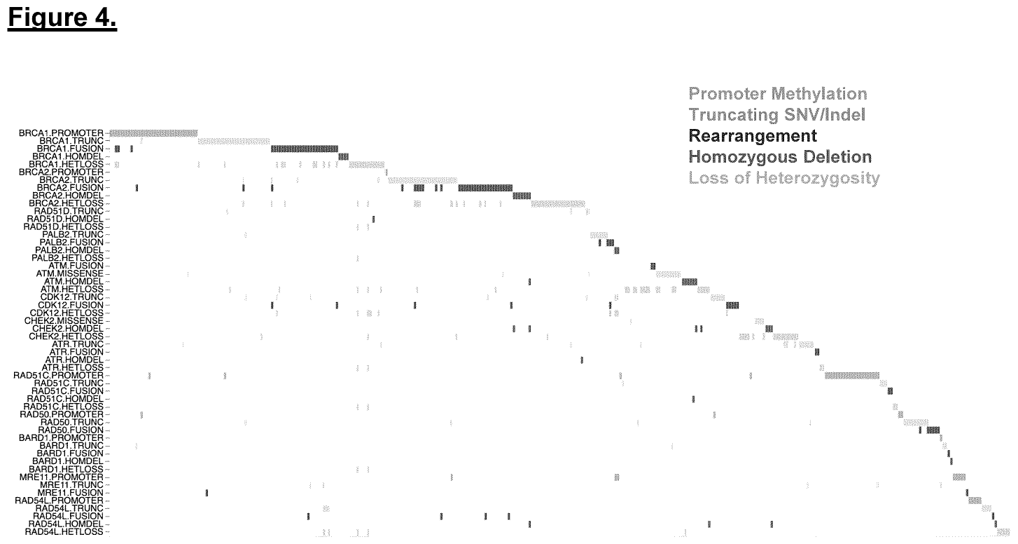

FIG. 5. Characteristics of promoter coverage in sample panel. Depicted herein is a minimal 10 CpG in 200 bp sliding window.

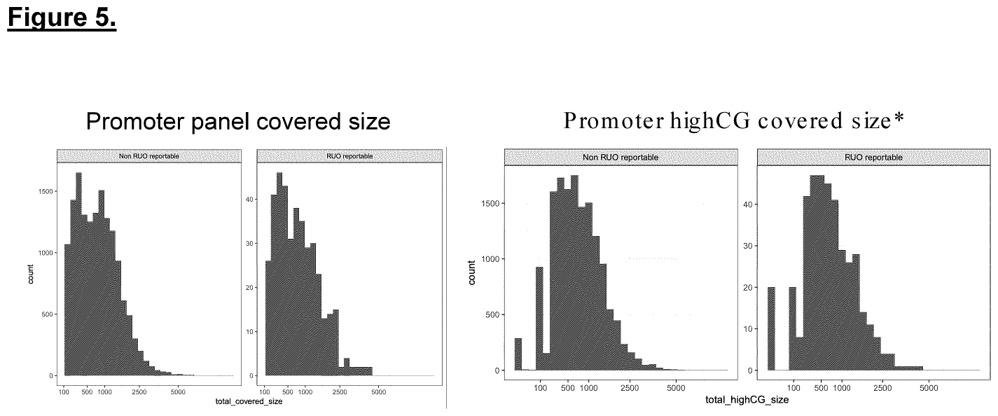

FIG. 6. Promoter methylation region definition Region removal: sex chromosome+normal noisy region. Aggregate 5kb upstream—TSS per gene. If multi TSS, aggregate at gene level. Definition approaches: Split by each TSS->report at transcript level; Refine promoter region through literature, other data (e.g. MBD partition peak, RNA/methylation associations). At least 2 probes in the promoter region for virtually all of 16,000 genes covered in a panel.

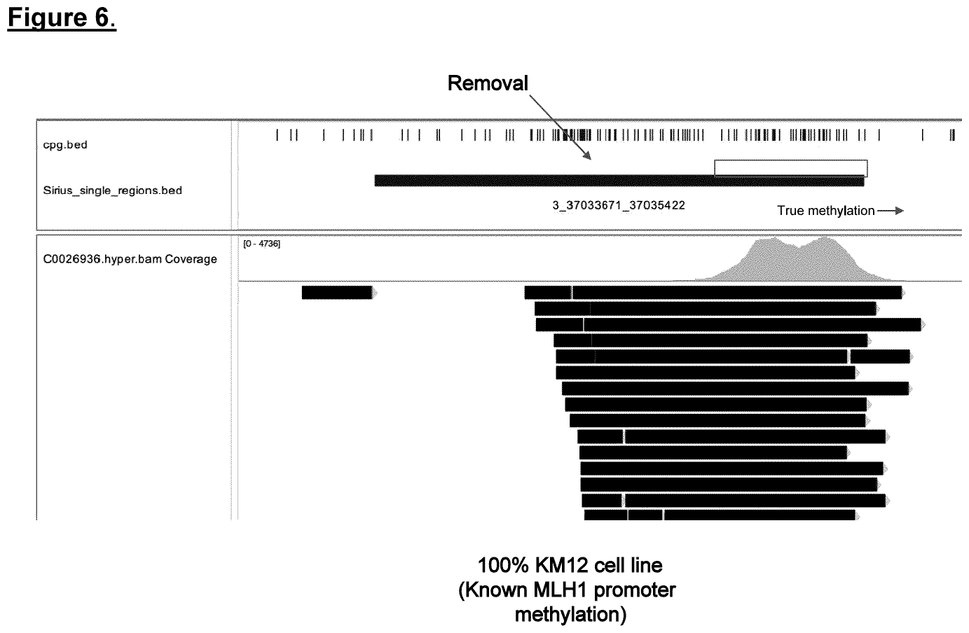

FIG. 7. Illustrative Analytical Validation: Limit of Detection.

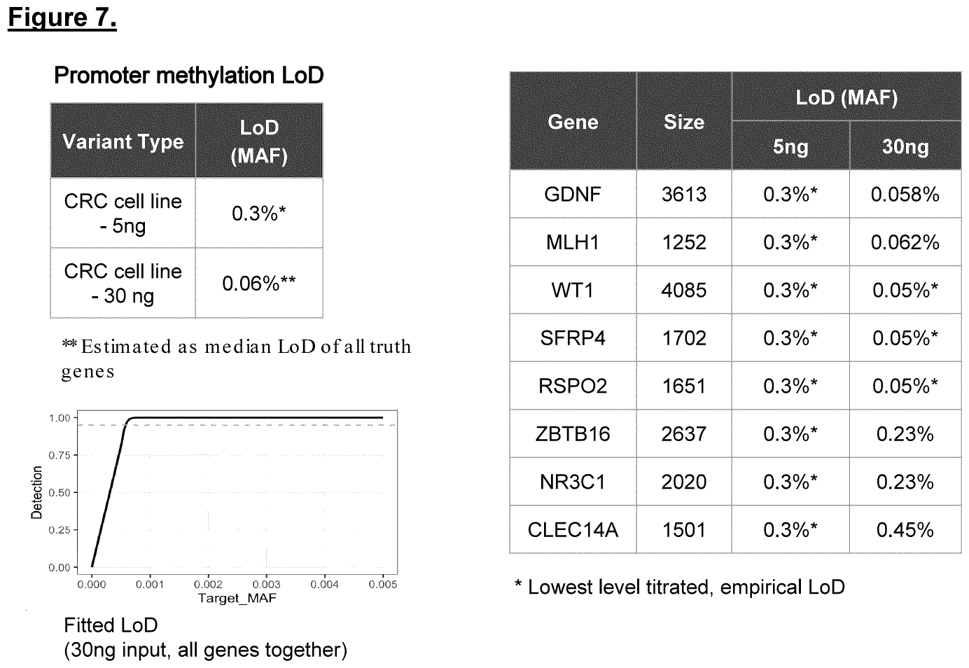

FIG. 8 Epigenomic MLH1 vs MSI-H association, MSI Promoter Definition.

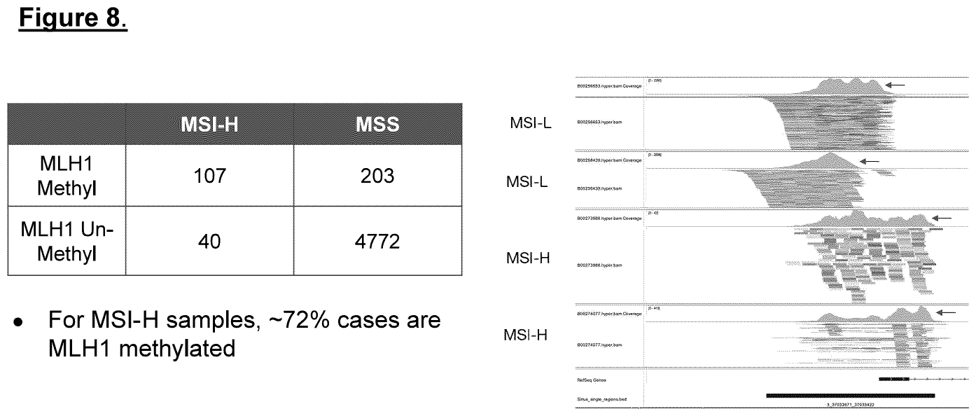

FIG. 9. Region pattern for MSI-H vs MSS/MSI-L for MLH1+.

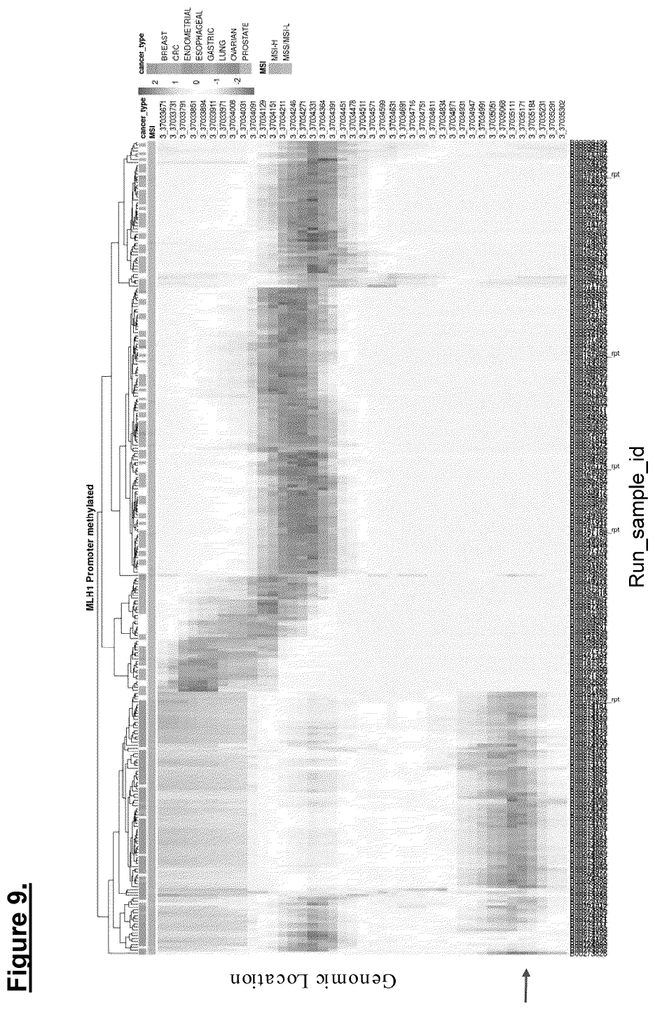

FIG. 10. BRCA1—clinical samples and cell lines.

FIG. 11. Promoter methylation: partial vs full methylation. In some instances, only full methylation can lead to gene inactivation. Promoter methylation often occurs in one allele while the other allele inactivated by other events. (e.g. BRCA LoH/Promoter co-occur in HRD+). Functional methylation changes may include differentiate partial vs full methylation.

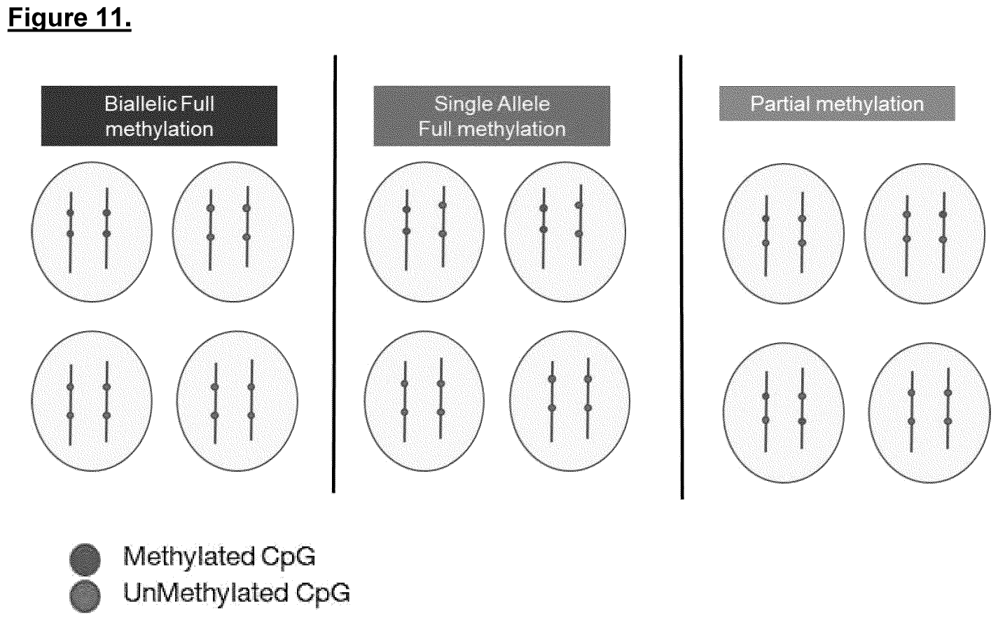

FIG. 12. EM-seq overview: panel design. In an orthogonal method to demonstrate capabilities of the detection scheme, a EM-seq panel was designed for pan-cancer methylation enrichment. Targets 1.54 Mb (125,080 CpGs)@15,000× depth with 13,090 probes. 1.00 Mb and 90,949 CpGs are covered by epigenomic probes (65% of sequence, 73% of CpGs); 876 kb and 70,493 of those CpGs overlap refseq promoter regions and MLH1 and BRCA1 shown.

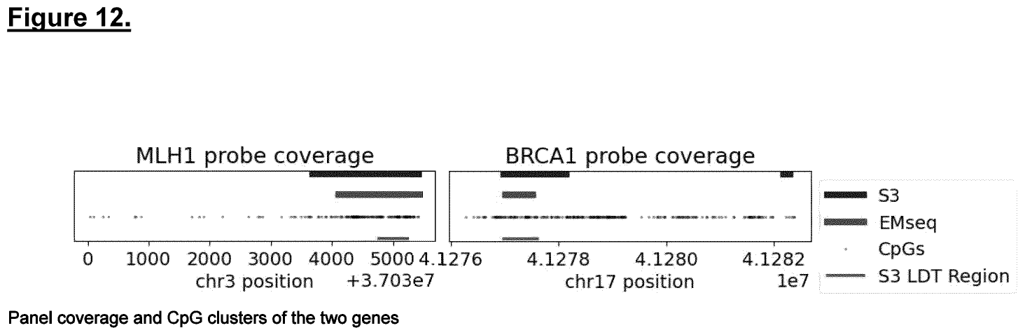

FIG. 13. EM-seq data consistency with public array data. Accuracy of orthogonal EM-seq results using neat cell lines is depicted. Variant-level (left) and probe-level (right) betas agree between KM12 EM-seq (x-axis) and Illumina 450K array data (NCI, y-axis). Probe beta is the mean of all CpG betas in each EM-seq region (both datasets).

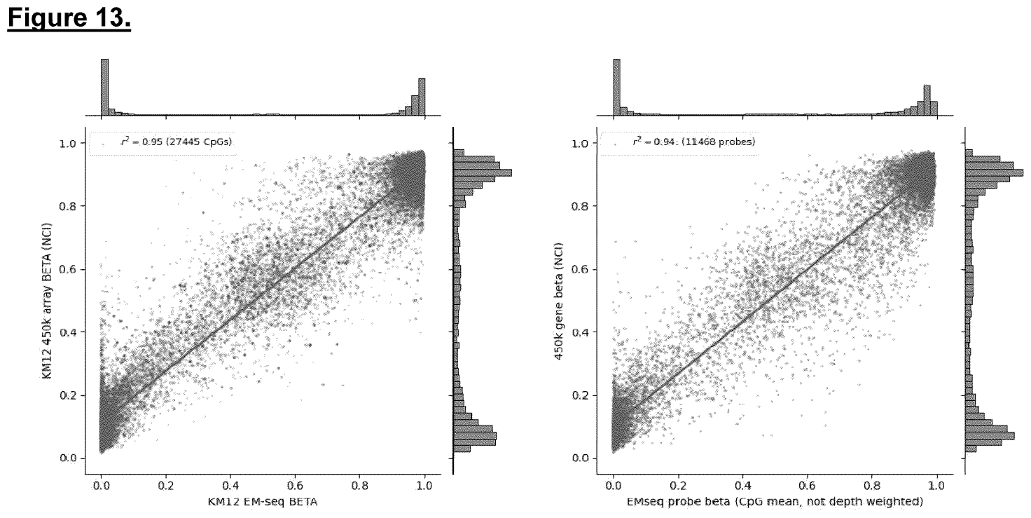

FIG. 14. Epigenomic detection region-wise TF vs EM-Seq region-wise. Here the accuracy of Positive Prediction Accuracy (PPA) in samples>=Epigenomic LoD (rough estimate>0.3% TF=red box on left plot). One can expect >80% PPA for EM-Seq because most of the samples have >0.1% beta value (calling threshold). Positive clinical samples with mixture of positive and negative promoter methylation calls across all genes on the epigenomic detection and EMSeq panels. Negative (cancer-free) clinical samples with mostly negative promoter methylation calls across genes on the epigenomic detection and EMSeq panels.

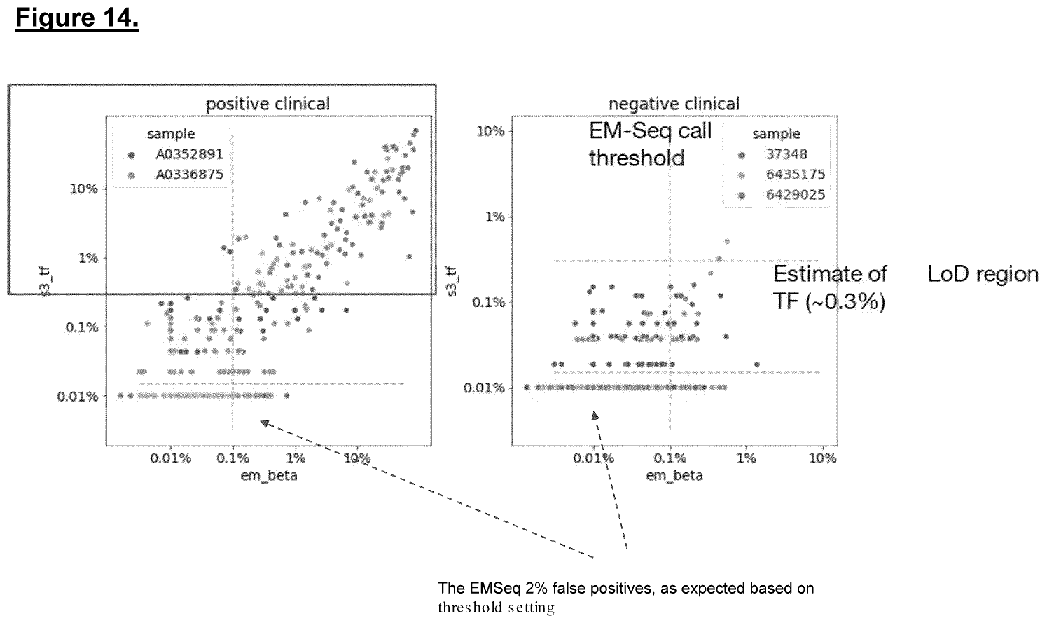

### DETAILED DESCRIPTION

BRCA1 promoter methylation (PM) is an early initiating event in cancer, occurring in 3 to 65.2% of all breast tumors depending on subtype, and 30 to 65% of triple negative tumors. BRCA1 promoter methylation has been associated with defective homologous recombination repair (HRR), early onset of breast and ovarian cancer, and improved clinical response to adjuvant chemotherapy. To date, there has been no diagnostic assay that comprehensively evaluates both BRCA1 promoter methylation and genomic alterations in cell-free circulating tumor DNA (ctDNA). Here, the Inventors have established a hereto unachieved detection method for interrogating both promoter methylation status, genomic alterations and further, quantification of methylation without or without epigenetic allelic status. Here, this multimodal detection of BRCA1 PM and genomic alterations in a cohort of patients with breast cancer using an epigenomic detection platform including methyl binding domain partitioning, allows a liquid biopsy assay interrogating 800+ genes and genome-wide methylation detection. Assessment for BRCA1 PM in ctDNA from 1016 patients with late-stage breast cancer was performed, along with genomic sequencing of 800+ genes and PM profiling of 398 cancer-related genes was performed by the epigenomic methylation detection assay. Pre-defined promoter regions of each covered gene were analyzed, including new approaches for promoter definition. For each sample, methylation scores were calculated for each gene and used as the basis for making PM calls. The limit of detection (LoD) was determined through in silico and experimental titrations of ctDNA from clinical samples and cell lines with known gene PM into the plasma of cancer-free donors.

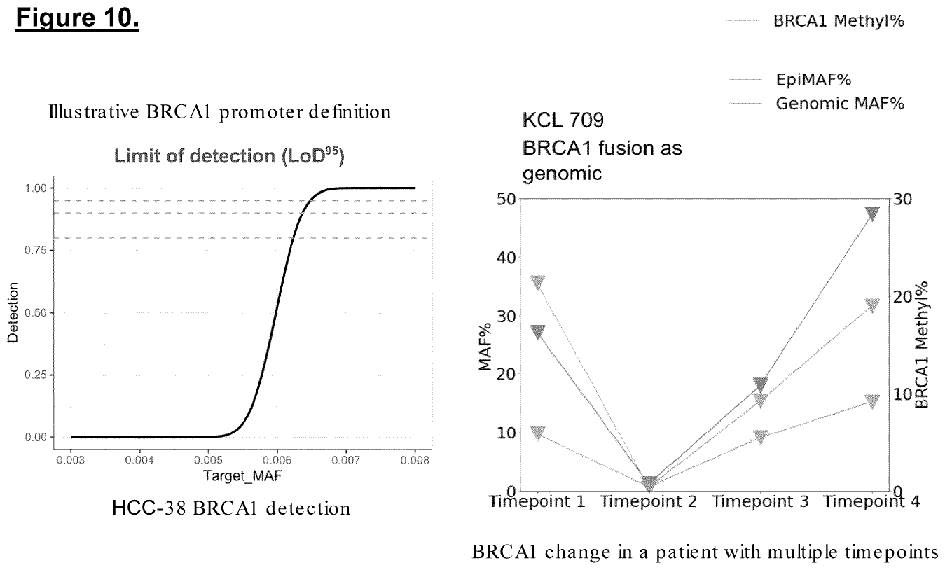

Additionally, establishing the aforementioned detection approach, allows epigenetic allelic status determination at a systematic-level. Allele-specific methylation patterns play an important role in controlling gene expression and maintaining normal cellular functions, and disruptions in these patterns can contribute to pathogenesis including oncogenesis. Imprinting is a form of allele-specific methylation pattern in which one allele of a gene is methylated and silenced depending on whether it is inherited from the mother or the father. The differential methylation and resulting monoallelic expression of imprinted genes are important for normal development and physiological functions and abnormal changes in these imprinting patterns (either loss or gain of methylation), can lead to developmental disorders and increased susceptibility to diseases, including cancer. For example, loss of imprinting (LOI) can lead to the expression of both alleles of a gene that is normally imprinted, potentially doubling the expression of genes that promote cell growth, a common feature in various cancers, see for example FIG. 10 panel A. In additional cases, tumor suppressor genes that are typically unmethylated and active can become methylated on one allele. This methylation can silence the gene's expression from that allele, contributing to cancer progression if the other allele is lost or mutated. A well-known example is the p16 gene (CDKN2A), which can undergo hypermethylation in various cancers such as melanoma, bladder cancer, and others, see for example FIG. 10 panel B. Partial allele-specific methylation patterns (see FIG. 10 panel C) may impact gene function more subtly compared to the complete methylation of an entire allele. This selective methylation can occur in specific regions of a gene, such as promoters, enhancers, or other regulatory elements, influencing the transcriptional activity of that gene in a cell-type specific manner. In cancer, partial methylation of promoter regions of tumor suppressor genes can downregulate gene expression without completely silencing the gene. This partial methylation might occur in only certain CpG islands within the promoter region. Additionally, methylation of enhancer regions can modulate the activity of enhancers, thus indirectly influencing the expression of genes associated with these enhancers. Partial methylation of enhancer regions can result in altered gene expression profiles that contribute to oncogenesis.

Current approaches are to omit testing both genomic and epigenomic attributes of the patient sample or to perform multiple tests separately. Omitting genomic or epigenomic information can result in prescription of cancer therapies that could be known to be ineffective or withholding cancer therapies that could be known to be effective, had both genomic and epigenomic information been available. For instance, patients with the KRASG12C biomarker are prescribed KRAS inhibitors but if epigenomic information showing the KRAS promoter was methylated and thus the gene silenced it would be apparent that KRAS inhibitors will not be effective. On the other hand, patients with no detected BRCA1 mutations may not be prescribed PARP inhibitors but if epigenomic information showing the BRCA1 promoter was methylated and thus the gene silenced the patient would be a good candidate for PARP inhibitors. Multiple tests are often not performed due to a variety of reasons including lack of sufficient patient samples. Other drawbacks include lack of reimbursement, inconvenience and lack of available commercial offerings etc.

Cancer can be indicated by epigenetic variations, such as methylation. Examples of methylation changes in cancer include local gains of DNA methylation in the CpG islands at the transcription start site (TSS) of genes involved in normal growth control, DNA repair, cell cycle regulation, and/or cell differentiation. This hypermethylation can be associated with an aberrant loss of transcriptional capacity of involved genes and occurs at least as frequently as point mutations and deletions as a cause of altered gene expression. DNA methylation profiling can be used to detect regions with different extents of methylation (“differentially methylated regions” or “DMRs”) of the genome that are altered during development or that are perturbed by disease, for example, cancer or any cancer-associated disease. The genome of cancer cells harbor imbalance in the above DNA methylation patterns, and therefore in functional packaging of the DNA. The abnormalities of chromatin organization are therefore coupled with methylation changes and may contribute to enhanced cancer profiling when analyzed jointly. Combining MBD-partitioning with fragmentomic data, such as fragment mapped starts and stops positions (correlated with nucleosome positions), fragment length and associated nucleosome occupancy, can be used for chromatin structure analysis in hypermethylation studies with the aim to improve biomarker detection rate.

Methylation profiling can involve determining methylation patterns across different regions of the genome. For example, after partitioning molecules based on extent of methylation (e.g., relative number of methylated sites per molecule) and sequencing, the sequences of molecules in the different partitions can be mapped to a reference genome. This can show regions of the genome that, compared with other regions, are more highly methylated or are less highly methylated. In this way, genomic regions, in contrast to individual molecules, may differ in their extent of methylation.

A characteristic of nucleic acid molecules may be a modification, which may include various chemical or protein modifications (i.e. epigenetic modifications). Non-limiting examples of chemical modification may include, but are not limited to, covalent DNA modifications, including DNA methylation. In some embodiments, DNA methylation includes addition of a methyl group to a cytosine at a CpG site (a cytosine followed by a guanine in a nucleic acid sequence). In some embodiments, DNA methylation includes addition of a methyl group to adenine, such as in N6-methyladenine. In some embodiments, DNA methylation is 5-methylation (modification of the 5th carbon of the 6 carbon ring of cytosine). In some embodiments, 5-methylation includes addition of a methyl group to the 5C position of the cytosine to create 5-methylcytosine (m5c). In some embodiments, methylation includes a derivative of m5c. Derivatives of m5c include, but are not limited to, 5-hydroxymethylcytosine (5-hmC), 5-formylcytosine (5-fC), and 5-caryboxylcytosine (5-caC). In some embodiments, DNA methylation is 3C methylation (modification of the 3rd carbon of the 6 carbon ring of cytosine). In some embodiments, 3C methylation includes addition of a methyl group to the 3C position of the cytosine to generate 3-methylcytosine (3mC). Other examples include N6-methyladenine or glycosylation. DNA methylation includes addition of methyl groups to DNA (e.g. CpG) and can change the expression of methylated DNA region. Methylation can also occur at non CpG sites, for example, methylation can occur at a CpA, CpT, or CpC site. DNA methylation can change the activity of methylated DNA region. For example, when DNA in a promoter region is methylated, transcription of the gene may be repressed. DNA methylation is critical for normal development and abnormality in methylation may disrupt epigenetic regulation. The disruption, e.g., repression, in epigenetic regulation may cause diseases, such as cancer. Promoter methylation in DNA may be indicative of cancer.

A CpG dyad is the dinucleotide CpG (cytosine-phosphate-guanine, i.e. a cytosine followed by a guanine in a 5′→3′ direction of the nucleic acid sequence) on the sense strand and its complementary CpG on the antisense strand of a double-stranded DNA molecule. CpG dyads can be either fully methylated or hemi-methylated (methylated on one strand only). The CpG dinucleotide is underrepresented in the normal human genome, with the majority of CpG dinucleotide sequences being transcriptionally inert (e.g. DNA heterochromatic regions in pericentromeric parts of the chromosome and in repeat elements) and methylated. However, many CpG islands are protected from such methylation especially around transcription start sites (TSS).

Protein modifications include binding to components of chromatin, particularly histones including modified forms thereof, and binding to other proteins, such as proteins involved in replication or transcription. The disclosure provides methods of processing and analyzing nucleic acids with different extents of modification, such that the nature of their original modification is correlated with a nucleic acid tag and can be decoded by sequencing the tag when nucleic acids are analyzed. Genetic variation of sample nucleic acid modifications can then be associated with the extent of modification (epigenetic variation) of that nucleic acid in the original sample. include single stranded (e.g., ssDNA or RNA) or double stranded molecules (e.g., dsDNA).

The loss of DNA can reduce the presence of one or more types of DNA such that the presence of the one or more types of DNA such as cfDNA, is difficult to detect. In one or more additional scenarios, existing methods to measure DNA methylation, such as enrichment or depletion methods, can have a relatively high level of resolution, such as about 100 base pairs (bp) to about 200 bp that can make accurately determining an amount of methylation of DNA difficult. The accuracy with which DNA methylation is determined can impact the accuracy of estimates of tumor fraction for samples. Since tumor fraction can be used to determine whether a sample is derived from a subject in which a tumor is present or not, the accuracy of determinations of tumor fraction estimates can impact diagnosis and/or treatment decisions for individuals.

More specifically, the techniques described herein allow quantification of promoter region methylation. Gedatolisib is an intravenously administered PI3K and mTOR inhibitor which has been shown to be safe in patients with metastatic breast cancer, either alone or in combination with oral therapies. Previous research has shown that PI3K inhibitors lower nucleotide pools required for DNA synthesis and S-phase progression. Additionally, inhibition of PI3K/mTOR could impede PI3K interaction with the homologous recombination complex, increasing dependency on PARP enzymes for DNA repair. Based on this data, the combination of a PI3K inhibitor and PARP inhibitor could potentially lead to a new, non-chemotherapy treatment option for TNBC with wild-type BRCA and improve the modest PFS seen with the PARP inhibitors as single agents in BRCA1/2 mutant advanced breast cancer. The hypothesis for this trial is that the gedatolisib will sensitize advanced TNBC or BRCA1/2 mutant breast cancers to PARP inhibition with talazoparib. Of interesting is determining the recommended phase 2 dose of gedatolisib in combination with talazoparib and to evaluate the efficacy of this combination in advanced HER2 negative breast cancer that is triple negative or BRCA1/2 positive (mutated/deficient).

### Samples

A sample can be any biological sample isolated from a subject. A sample can be a bodily sample. Samples can include body tissues, such as known or suspected solid tumors, whole blood, platelets, serum, plasma, stool, red blood cells, white blood cells or leucocytes, endothelial cells, tissue biopsies, cerebrospinal fluid synovial fluid, lymphatic fluid, ascites fluid, interstitial or extracellular fluid, the fluid in spaces between cells, including gingival crevicular fluid, bone marrow, pleural effusions, cerebrospinal fluid, saliva, mucous, sputum, semen, sweat, urine. Samples are preferably body fluids, particularly blood and fractions thereof, and urine. A sample can be in the form originally isolated from a subject or can have been subjected to further processing to remove or add components, such as cells, or enrich for one component relative to another. Thus, a preferred body fluid for analysis is plasma or serum containing cell-free nucleic acids. A sample can be isolated or obtained from a subject and transported to a site of sample analysis. The sample may be preserved and shipped at a desirable temperature, e.g., room temperature, 4° C., −20° C., and/or −80° C. A sample can be isolated or obtained from a subject at the site of the sample analysis. The subject can be a human, a mammal, an animal, a companion animal, a service animal, or a pet. The subject may have a cancer. The subject may not have cancer or a detectable cancer symptom. The subject may have been treated with one or more cancer therapy, e.g., any one or more of chemotherapies, antibodies, vaccines or biologics. The subject may be in remission. The subject may or may not be diagnosed of being susceptible to cancer or any cancer-associated genetic mutations/disorders.

The volume of plasma can depend on the desired read depth for sequenced regions. Exemplary volumes are 0.4-40 ml, 5-20 ml, 10-20 ml. For examples, the volume can be 0.5 mL, 1 mL, 5 mL 10 mL, 20 mL, 30 mL, or 40 mL. A volume of sampled plasma may be 5 to 20 mL.

A sample can comprise various amount of nucleic acid that contains genome equivalents. For example, a sample of about 30 ng DNA can contain about 10,000 (104) haploid human genome equivalents and, in the case of cfDNA, about 200 billion (2×1011) individual polynucleotide molecules. Similarly, a sample of about 100 ng of DNA can contain about 30,000 haploid human genome equivalents and, in the case of cfDNA, about 600 billion individual molecules.

A sample can comprise nucleic acids from different sources, e.g., from cells and cell-free of the same subject, from cells and cell-free of different subjects. A sample can comprise nucleic acids carrying mutations. For example, a sample can comprise DNA carrying germline mutations and/or somatic mutations. Germline mutations refer to mutations existing in germline DNA of a subject. Somatic mutations refer to mutations originating in somatic cells of a subject, e.g., cancer cells. A sample can comprise DNA carrying cancer-associated mutations (e.g., cancer-associated somatic mutations). A sample can comprise an epigenetic variant (i.e. a chemical or protein modification), wherein the epigenetic variant associated with the presence of a genetic variant such as a cancer-associated mutation. In some embodiments, the sample includes an epigenetic variant associated with the presence of a genetic variant, wherein the sample does not comprise the genetic variant.

Exemplary amounts of cell-free nucleic acids in a sample before amplification range from about 1 fg to about 1 μg, e.g., 1 pg to 200 ng, 1 ng to 100 ng, 10 ng to 1000 ng. For example, the amount can be up to about 600 ng, up to about 500 ng, up to about 400 ng, up to about 300 ng, up to about 200 ng, up to about 100 ng, up to about 50 ng, or up to about 20 ng of cell-free nucleic acid molecules. The amount can be at least 1 fg, at least 10 fg, at least 100 fg, at least 1 pg, at least 10 pg, at least 100 pg, at least 1 ng, at least 10 ng, at least 100 ng, at least 150 ng, or at least 200 ng of cell-free nucleic acid molecules. The amount can be up to 1 femtogram (fg), 10 fg, 100 fg, 1 picogram (pg), 10 pg, 100 pg, 1 ng, 10 ng, 100 ng, 150 ng, or 200 ng of cell-free nucleic acid molecules. The method can comprise obtaining 1 femtogram (fg) to 200 ng.

Cell-free nucleic acids are nucleic acids not contained within or otherwise bound to a cell or in other words nucleic acids remaining in a sample after removing intact cells. Cell-free nucleic acids include DNA, RNA, and hybrids thereof, including genomic DNA, mitochondrial DNA, siRNA, miRNA, circulating RNA (cRNA), tRNA, rRNA, small nucleolar RNA (snoRNA), Piwi-interacting RNA (piRNA), long non-coding RNA (long ncRNA), or fragments of any of these. Cell-free nucleic acids can be double-stranded, single-stranded, or a hybrid thereof. A cell-free nucleic acid can be released into bodily fluid through secretion or cell death processes, e.g., cellular necrosis and apoptosis. Some cell-free nucleic acids are released into bodily fluid from cancer cells e.g., circulating tumor DNA, (ctDNA). Others are released from healthy cells. In some embodiments, cfDNA is cell-free fetal DNA (cffDNA) In some embodiments, cell free nucleic acids are produced by tumor cells. In some embodiments, cell free nucleic acids are produced by a mixture of tumor cells and non-tumor cells.

Cell-free nucleic acids have an exemplary size distribution of about 100-500 nucleotides, with molecules of 110 to about 230 nucleotides representing about 90% of molecules, with a mode of about 168 nucleotides and a second minor peak in a range between 240 to 440 nucleotides. Cell-free nucleic acids can be isolated from bodily fluids through a fractionation or partitioning step in which cell-free nucleic acids, as found in solution, are separated from intact cells and other non-soluble components of the bodily fluid. Partitioning may include techniques such as centrifugation or filtration. Alternatively, cells in bodily fluids can be lysed and cell-free and cellular nucleic acids processed together. Generally, after addition of buffers and wash steps, nucleic acids can be precipitated with an alcohol. Further clean up steps may be used such as silica based columns to remove contaminants or salts. Non-specific bulk carrier nucleic acids, such as Cot-1 DNA, DNA or protein for bisulfite sequencing, hybridization, and/or ligation, may be added throughout the reaction to optimize certain aspects of the procedure such as yield.

After such processing, samples can include various forms of nucleic acid including double stranded DNA, single stranded DNA and single stranded RNA. In some embodiments, single stranded DNA and RNA can be converted to double stranded forms so they are included in subsequent processing and analysis steps.

### Analytes

Analytes can include nucleic acid analytes, and non-nucleic acid analytes. The disclosure provides for detecting genetic variations in biological samples from a subject. Biological samples may include polynucleotides from cancer cells. Polynucleotides may be DNA (e.g., genomic DNA, cDNA), RNA (e.g., mRNA, small RNAs), or any combination thereof. Biological samples may include tumor tissue, e.g., from a biopsy. In some cases, biological samples may include blood or saliva. In particular cases, biological samples may comprise cell free DNA (“cfDNA”) or circulating tumor DNA (“ctDNA”). Cell free DNA can be present in, e.g., blood.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquity lati on variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. This further includes receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

In general, the systems, apparatus, methods, and compositions can be used to analyze any number of analytes, further including both nucleic acid analytes and non-nucleic acid analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate. Methods for performing multiplexed assays to analyze two or more different analytes will be discussed in a subsequent section of this disclosure.

One or more nucleic acid analytes and/or non-nucleic acid analytes constitute a set of molecular interactions in a biological system under study (e.g., cells), which may be regarded as “interactome”—the molecular interactions that occur between molecules belonging to different biochemical families (proteins, nucleic acids, lipids, carbohydrates, etc.) and also within a given family. In various embodiments, an interactome is a protein-DNA interactome (network formed by transcription factors (and DNA or chromatin regulatory proteins) and their target genes. In other embodiments, interactome refers to protein-protein interaction network (PPI), or protein interaction network (PIN). The methods described herein allow for study and analysis of the interactome. Techniques such as proteogenomics (whole genome sequencing, whole exome sequencing and RNA-seq, and mass spectrometry as examples) can support study of the interactome.

### Analysis

The present methods can be used to diagnose presence of conditions, particularly cancer, in a subject, to characterize conditions (e.g., staging cancer or determining heterogeneity of a cancer), monitor response to treatment of a condition, effect prognosis risk of developing a condition or subsequent course of a condition. The present disclosure can also be useful in determining the efficacy of a particular treatment option. Successful treatment options may increase the amount of copy number variation or rare mutations detected in subject's blood if the treatment is successful as more cancers may die and shed DNA. In other examples, this may not occur. In another example, perhaps certain treatment options may be correlated with genetic profiles of cancers over time. This correlation may be useful in selecting a therapy. Additionally, if a cancer is observed to be in remission after treatment, the present methods can be used to monitor residual disease or recurrence of disease.

The types and number of cancers that may be detected may include blood cancers, brain cancers, lung cancers, skin cancers, nose cancers, throat cancers, liver cancers, bone cancers, lymphomas, pancreatic cancers, skin cancers, bowel cancers, rectal cancers, thyroid cancers, bladder cancers, kidney cancers, mouth cancers, stomach cancers, solid state tumors, heterogeneous tumors, homogenous tumors and the like. Type and/or stage of cancer can be detected from genetic variations including mutations, rare mutations, indels, copy number variations, transversions, translocations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine.

Genetic and other analyte data can also be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

The present analyses are also useful in determining the efficacy of a particular treatment option. Successful treatment options may increase the amount of copy number variation or rare mutations detected in subject's blood if the treatment is successful as more cancers may die and shed DNA. In other examples, this may not occur. In another example, perhaps certain treatment options may be correlated with genetic profiles of cancers over time. This correlation may be useful in selecting a therapy. Additionally, if a cancer is observed to be in remission after treatment, the present methods can be used to monitor residual disease or recurrence of disease.

The present methods can also be used for detecting genetic variations in conditions other than cancer. Immune cells, such as B cells, may undergo rapid clonal expansion upon the presence certain diseases. Clonal expansions may be monitored using copy number variation detection and certain immune states may be monitored. In this example, copy number variation analysis may be performed over time to produce a profile of how a particular disease may be progressing. Copy number variation or even rare mutation detection may be used to determine how a population of pathogens is changing during the course of infection. This may be particularly important during chronic infections, such as HIV/AIDS or Hepatitis infections, whereby viruses may change life cycle state and/or mutate into more virulent forms during the course of infection. The present methods may be used to determine or profile rejection activities of the host body, as immune cells attempt to destroy transplanted tissue to monitor the status of transplanted tissue as well as altering the course of treatment or prevention of rejection.

Further, the methods of the disclosure may be used to characterize the heterogeneity of an abnormal condition in a subject. Such methods can include, e.g., generating a genetic profile of extracellular polynucleotides derived from the subject, wherein the genetic profile includes a plurality of data resulting from copy number variation and rare mutation analyses. In some embodiments, an abnormal condition is cancer. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease. Again, in the example of cancer, there may be multiple tumor foci, perhaps where one or more foci are the result of metastases that have spread from a primary site.

The present methods can be used to generate or profile, fingerprint or set of data that is a summation of genetic information derived from different cells in a heterogeneous disease. This set of data may comprise copy number variation and mutation analyses alone or in combination.

The present methods can be used to diagnose, prognose, monitor or observe cancers. or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

### Determination of 5-Methylcytosine Pattern of Nucleic Acids

Bisulfite-based sequencing and variants thereof provides a means of determining the methylation pattern of a nucleic acid. In some embodiments, determining the methylation pattern comprises distinguishing 5-methylcytosine (5mC) from non-methylated cytosine. In some embodiments, determining methylation pattern comprises distinguishing N6-methyladenine from non-methylated adenine. In some embodiments, determining the methylation pattern comprises distinguishing 5-hydroxymethylcytosine (5hmC), 5-formylcytosine (5fC), and 5-carboxylcytosine (5caC) from non-methylated cytosine. Examples of bisulfite sequencing include, but are not limited to oxidative bisulfite sequencing (OX-BS-seq), Tet-assisted bisulfite sequencing (TAB-seq), and reduced bisulfite sequencing (redBS-seq).

Oxidative bisulfite sequencing (OX-BS-seq) is used to distinguish between 5mC and 5hmC, by first converting the 5hmC to 5fC, and then proceeding with bisulfite sequencing as previously described. Tet-assisted bisulfite sequencing (TAB-seq) can also be used to distinguish 5mc and 5hmC. In TAB-seq, 5hmC is protected by glucosylation. A Tet enzyme is then used to convert 5mC to 5caC before proceeding with bisulfite sequencing, as previously described. Reduced bisulfite sequencing is used to distinguish 5fC from modified cytosines.

Generally, in bisulfite sequencing, a nucleic acid sample is divided into two aliquots and one aliquot is treated with bisulfite. The bisulfite converts native cytosine and certain modified cytosine nucleotides (e.g. 5-formylcytosine or 5-carboxylcytosine) to uracil whereas other modified cytosines (e.g., 5-methylcytosine, 5-hydroxylmethylcystosine) are not converted. Comparison of nucleic acid sequences of molecules from the two aliquots indicates which cytosines were and were not converted to uracils. Consequently, cytosines which were and were not modified can be determined. The initial splitting of the sample into two aliquots is disadvantageous for samples containing only small amounts of nucleic acids, and/or composed of heterogeneous cell/tissue origins such as bodily fluids containing cell-free DNA.

The present disclosure provides methods allowing bisulfite sequencing and variants thereof. These methods work by linking nucleic acids in a population to a capture moiety, i.e., a label that can be captured or immobilized. Capture moieties include, without limitation, biotin, avidin, streptavidin, a nucleic acid comprising a particular nucleotide sequence, a hapten recognized by an antibody, and magnetically attractable particles. The extraction moiety can be a member of a binding pair, such as biotin/streptavidin or hapten/antibody. In some embodiments, a capture moiety that is attached to an analyte is captured by its binding pair which is attached to an isolatable moiety, such as a magnetically attractable particle or a large particle that can be sedimented through centrifugation. The capture moiety can be any type of molecule that allows affinity separation of nucleic acids bearing the capture moiety from nucleic acids lacking the capture moiety. Exemplary capture moieties are biotin which allows affinity separation by binding to streptavidin linked or linkable to a solid phase or an oligonucleotide, which allows affinity separation through binding to a complementary oligonucleotide linked or linkable to a solid phase. Following linking of capture moieties to sample nucleic acids, the sample nucleic acids serve as templates for amplification. Following amplification, the original templates remain linked to the capture moieties but amplicons are not linked to capture moieties.

The capture moiety can be linked to sample nucleic acids as a component of an adapter, which may also provide amplification and/or sequencing primer binding sites. In some methods, sample nucleic acids are linked to adapters at both ends, with both adapters bearing a capture moiety. Preferably any cytosine residues in the adapters are modified, such as by 5methylcytosine, to protect against the action of bisulfite. In some instances, the capture moieties are linked to the original templates by a cleavable linkage (e.g., photocleavable desthiobiotin-TEG or uracil residues cleavable with USER™ enzyme, Chem. Commun. (Camb). 2015 Feb. 21; 51(15): 3266-3269), in which case the capture moieties can, if desired, be removed.

The amplicons are denatured and contacted with an affinity reagent for the capture tag. Original templates bind to the affinity reagent whereas nucleic acid molecules resulting from amplification do not. Thus, the original templates can be separated from nucleic acid molecules resulting from amplification.

Following separation or partition, the respective populations of nucleic acids (i.e., original templates and amplification products) can be subjected to bisulfite treatment with the original template population receiving bisulfite treatment and the amplification products not. Alternatively, the amplification products can be subjected to bisulfite treatment and the original template population not. Following such treatment, the respective populations can be amplified (which in the case of the original template population converts uracils to thymines). The populations can also be subjected to biotin probe hybridization for enrichment. The respective populations are then analyzed and sequences compared to determine which cytosines were 5-methylated (or 5-hydroxylmethylated) in the original. Detection of a T nucleotide in the template population (corresponding to an unmethylated cytosine converted to uracil) and a C nucleotide at the corresponding position of the amplified population indicates an unmodified C. The presence of C's at corresponding positions of the original template and amplified populations indicates a modified C in the original sample.

In some embodiments, a method uses sequential DNA-seq and bisulfite-seq (BIS-seq) NGS library preparation of molecular tagged DNA libraries. This process is performed by labeling of adapters (e.g., biotin), DNA-seq amplification of whole library, parent molecule recovery (e.g. streptavidin bead pull down), bisulfite conversion and BIS-seq. In some embodiments, the method identifies 5-methylcytosine with single-base resolution, through sequential NGS-preparative amplification of parent library molecules with and without bisulfite treatment. This can be achieved by modifying the 5-methyl-ated NGS-adapters (directional adapters; Y-shaped/forked with 5-methylcytosine replacing) used in BIS-seq with a label (e.g., biotin) on one of the two adapter strands. Sample DNA molecules are adapter ligated, and amplified (e.g., by PCR). As only the parent molecules will have a labeled adapter end, they can be selectively recovered from their amplified progeny by label-specific capture methods (e.g., streptavidin-magnetic beads). As the parent molecules retain 5-methylation marks, bisulfite conversion on the captured library will yield single-base resolution 5-methylation status upon BIS-seq, retaining molecular information to corresponding DNA-seq. In some embodiments, the bisulfite treated library can be combined with a non-treated library prior to enrichment/NGS by addition of a sample tag DNA sequence in standard multiplexed NGS workflow. As with BIS-seq workflows, bioinformatics analysis can be carried out for genomic alignment and 5-methylated base identification. In sum, this method provides the ability to selectively recover the parent, ligated molecules, carrying 5-methylcytosine marks, after library amplification, thereby allowing for parallel processing for bisulfite converted DNA. This overcomes the destructive nature of bisulfite treatment on the quality/sensitivity of the DNA-seq information extracted from a workflow. With this method, the recovered ligated, parent DNA molecules (via labeled adapters) allow amplification of the complete DNA library and parallel application of treatments that elicit epigenetic DNA modifications. The present disclosure discusses the use of BIS-seq methods to identify cytosine5-methylation (5-methylcytosine), but this should is not limiting. Variants of BIS-seq have been developed to identify hydroxymethylated cytosines (5hmC; OX-BS-seq, TAB-seq), formylcytosine (5fC; redBS-seq) and carboxylcytosines. These methodologies can be implemented with the sequential/parallel library preparation described herein.

### Alternative Methods of Modified Nucleic Acid Analysis

The disclosure provides alternative methods for analyzing modified nucleic acids (e.g., methylated, linked to histones and other modifications discussed above). In some such methods, a population of nucleic acids bearing the modification to different extents (e.g., 0, 1, 2, 3, 4, 5 or more methyl groups per nucleic acid molecule) is contacted with adapters before fractionation of the population depending on the extent of the modification. Adapters attach to either one end or both ends of nucleic acid molecules in the population. Preferably, the adapters include different tags of sufficient numbers that the number of combinations of tags results in a low probability e.g., 95, 99 or 99.9% of two nucleic acids with the same start and stop points receiving the same combination of tags. Following attachment of adapters, the nucleic acids are amplified from primers binding to the primer binding sites within the adapters. Adapters, whether bearing the same or different tags, can include the same or different primer binding sites, but preferably adapters include the same primer binding site. Following amplification, the nucleic acids are contacted with an agent that preferably binds to nucleic acids bearing the modification (such as the previously described such agents). The nucleic acids are separated into at least two partitions differing in the extent to which the nucleic acids bear the modification from binding to the agents. For example, if the agent has affinity for nucleic acids bearing the modification, nucleic acids overrepresented in the modification (compared with median representation in the population) preferentially bind to the agent, whereas nucleic acids underrepresented for the modification do not bind or are more easily eluted from the agent. Following separation, the different partitions can then be subject to further processing steps, which typically include further amplification, and sequence analysis, in parallel but separately. Sequence data from the different partitions can then be compared.

Nucleic acids can be linked at both ends to Y-shaped adapters including primer binding sites and tags. The molecules are amplified. The amplified molecules are then fractionated by contact with an antibody preferentially binding to 5-methylcytosine to produce two partitions. One partition includes original molecules lacking methylation and amplification copies having lost methylation. The other partition includes original DNA molecules with methylation. The two partitions are then processed and sequenced separately with further amplification of the methylated partition. The sequence data of the two partitions can then be compared. In this example, tags are not used to distinguish between methylated and unmethylated DNA but rather to distinguish between different molecules within these partitions so that one can determine whether reads with the same start and stop points are based on the same or different molecules.

The disclosure provides further methods for analyzing a population of nucleic acid in which at least some of the nucleic acids include one or more modified cytosine residues, such as 5-methylcytosine and any of the other modifications described previously. In these methods, the population of nucleic acids is contacted with adapters including one or more cytosine residues modified at the 5C position, such as 5-methylcytosine. Preferably all cytosine residues in such adapters are also modified, or all such cytosines in a primer binding region of the adapters are modified. Adapters attach to both ends of nucleic acid molecules in the population. Preferably, the adapters include different tags of sufficient numbers that the number of combinations of tags results in a low probability e.g., 95, 99 or 99.9% of two nucleic acids with the same start and stop points receiving the same combination of tags. The primer binding sites in such adapters can be the same or different, but are preferably the same. After attachment of adapters, the nucleic acids are amplified from primers binding to the primer binding sites of the adapters. The amplified nucleic acids are split into first and second aliquots. The first aliquot is assayed for sequence data with or without further processing. The sequence data on molecules in the first aliquot is thus determined irrespective of the initial methylation state of the nucleic acid molecules. The nucleic acid molecules in the second aliquot are treated with bisulfite. This treatment converts unmodified cytosines to uracils. The bisulfite treated nucleic acids are then subjected to amplification primed by primers to the original primer binding sites of the adapters linked to nucleic acid. Only the nucleic acid molecules originally linked to adapters (as distinct from amplification products thereof) are now amplifiable because these nucleic acids retain cytosines in the primer binding sites of the adapters, whereas amplification products have lost the methylation of these cytosine residues, which have undergone conversion to uracils in the bisulfite treatment. Thus, only original molecules in the populations, at least some of which are methylated, undergo amplification. After amplification, these nucleic acids are subject to sequence analysis. Comparison of sequences determined from the first and second aliquots can indicate among other things, which cytosines in the nucleic acid population were subject to methylation.

### Partitioning the Sample into a Plurality of Subsamples; Aspects of Samples; Analysis of Epigenetic Characteristics

In certain embodiments described herein, a population of different forms of nucleic acids (e.g., hypermethylated and hypomethylated DNA in a sample, such as a captured set of cfDNA as described herein) can be physically partitioned based on one or more characteristics of the nucleic acids prior to further analysis, e.g., differentially modifying or isolating a nucleobase, tagging, and/or sequencing. This approach can be used to determine, for example, whether certain sequences are hypermethylated or hypomethylated. In some embodiments, hypermethylation variable epigenetic target regions are analyzed to determine whether they show hypermethylation characteristic of tumor cells and/or hypomethylation variable epigenetic target regions are analyzed to determine whether they show hypomethylation characteristic of tumor cells. Additionally, by partitioning a heterogeneous nucleic acid population, one may increase rare signals, e.g., by enriching rare nucleic acid molecules that are more prevalent in one fraction (or partition) of the population. For example, a genetic variation present in hyper-methylated DNA but less (or not) in hypomethylated DNA can be more easily detected by partitioning a sample into hyper-methylated and hypo-methylated nucleic acid molecules. By analyzing multiple fractions of a sample, a multi-dimensional analysis of a single locus of a genome or species of nucleic acid can be performed and hence, greater sensitivity can be achieved.

In some instances, a heterogeneous nucleic acid sample is partitioned into two or more partitions (e.g., at least 3, 4, 5, 6 or 7 partitions). In some embodiments, each partition is differentially tagged. Tagged partitions can then be pooled together for collective sample prep and/or sequencing. The partitioning-tagging-pooling steps can occur more than once, with each round of partitioning occurring based on a different characteristics (examples provided herein), and tagged using differential tags that are distinguished from other partitions and partitioning means.

Examples of characteristics that can be used for partitioning include sequence length, methylation level, nucleosome binding, sequence mismatch, immunoprecipitation, and/or proteins that bind to DNA. Resulting partitions can include one or more of the following nucleic acid forms: single-stranded DNA (ssDNA), double-stranded DNA (dsDNA), shorter DNA fragments and longer DNA fragments. In some embodiments, partitioning based on a cytosine modification (e.g., cytosine methylation) or methylation generally is performed and is optionally combined with at least one additional partitioning step, which may be based on any of the foregoing characteristics or forms of DNA. In some embodiments, a heterogeneous population of nucleic acids is partitioned into nucleic acids with one or more epigenetic modifications and without the one or more epigenetic modifications. Examples of epigenetic modifications include presence or absence of methylation; level of methylation; type of methylation (e.g., 5-methylcytosine versus other types of methylation, such as adenine methylation and/or cytosine hydroxymethylation); and association and level of association with one or more proteins, such as histones. Alternatively or additionally, a heterogeneous population of nucleic acids can be partitioned into nucleic acid molecules associated with nucleosomes and nucleic acid molecules devoid of nucleosomes. Alternatively or additionally, a heterogeneous population of nucleic acids may be partitioned into single-stranded DNA (ssDNA) and double-stranded DNA (dsDNA). Alternatively, or additionally, a heterogeneous population of nucleic acids may be partitioned based on nucleic acid length (e.g., molecules of up to 160 bp and molecules having a length of greater than 160 bp).

In some instances, each partition (representative of a different nucleic acid form) is differentially labelled, and the partitions are pooled together prior to sequencing. In other instances, the different forms are separately sequenced. In some embodiments, a population of different nucleic acids is partitioned into two or more different partitions. Each partition is representative of a different nucleic acid form, and a first partition (also referred to as a subsample) comprises DNA with a cytosine modification in a greater proportion than a second subsample. Each partition is distinctly tagged. The first subsample is subjected to a procedure that affects a first nucleobase in the DNA differently from a second nucleobase in the DNA of the first subsample, wherein the first nucleobase is a modified or unmodified nucleobase, the second nucleobase is a modified or unmodified nucleobase different from the first nucleobase, and the first nucleobase and the second nucleobase have the same base pairing specificity. The tagged nucleic acids are pooled together prior to sequencing. Sequence reads are obtained and analyzed, including to distinguish the first nucleobase from the second nucleobase in the DNA of the first subsample, in silico. Tags are used to sort reads from different partitions. Analysis to detect genetic variants can be performed on a partition-by-partition level, as well as whole nucleic acid population level. For example, analysis can include in silico analysis to determine genetic variants, such as CNV, SNV, indel, fusion in nucleic acids in each partition. In some instances, in silico analysis can include determining chromatin structure. For example, coverage of sequence reads can be used to determine nucleosome positioning in chromatin. Higher coverage can correlate with higher nucleosome occupancy in genomic region while lower coverage can correlate with lower nucleosome occupancy or nucleosome depleted region (NDR).

Samples can include nucleic acids varying in modifications including post-replication modifications to nucleotides and binding, usually noncovalently, to one or more proteins.

In an embodiment, the population of nucleic acids is one obtained from a serum, plasma or blood sample from a subject suspected of having neoplasia, a tumor, or cancer or previously diagnosed with neoplasia, a tumor, or cancer. The population of nucleic acids includes nucleic acids having varying levels of methylation. Methylation can occur from any one or more post-replication or transcriptional modifications. Post-replication modifications include modifications of the nucleotide cytosine, particularly at the 5-position of the nucleobase, e.g., 5-methylcytosine, 5-hydroxymethylcytosine, 5-formylcytosine and 5-carboxylcytosine. The affinity agents can be antibodies with the desired specificity, natural binding partners or variants thereof (Bock et al., Nat Biotech 28:1106-1114 (2010); Song et al., Nat Biotech 29:68-72 (2011)), or artificial peptides selected e.g., by phage display to have specificity to a given target.

Examples of capture moieties contemplated herein include methyl binding domain (MBDs) and methyl binding proteins (MBPs) as described herein, including proteins such as MeCP2 and antibodies preferentially binding to 5-methylcytosine. Likewise, partitioning of different forms of nucleic acids can be performed using histone binding proteins which can separate nucleic acids bound to histones from free or unbound nucleic acids. Examples of histone binding proteins that can be used in the methods disclosed herein include RBBP4, RbAp48 and SANT domain peptides. Although for some affinity agents and modifications, binding to the agent may occur in an essentially all or none manner depending on whether a nucleic acid bears a modification, the separation may be one of degree. In such instances, nucleic acids overrepresented in a modification bind to the agent at a greater extent that nucleic acids underrepresented in the modification. Alternatively, nucleic acids having modifications may bind in an all or nothing manner. But then, various levels of modifications may be sequentially eluted from the binding agent.

For example, in some embodiments, partitioning can be binary or based on degree/level of modifications. For example, all methylated fragments can be partitioned from unmethylated fragments using methyl-binding domain proteins (e.g., MethylMiner Methylated DNA Enrichment Kit (ThermoFisher Scientific)). Subsequently, additional partitioning may involve eluting fragments having different levels of methylation by adjusting the salt concentration in a solution with the methyl-binding domain and bound fragments. As salt concentration increases, fragments having greater methylation levels are eluted. In some instances, the final partitions are representative of nucleic acids having different extents of modifications (overrepresentative or underrepresentative of modifications). Overrepresentation and underrepresentation can be defined by the number of modifications born by a nucleic acid relative to the median number of modifications per strand in a population. For example, if the median number of 5-methylcytosine residues in nucleic acid in a sample is 2, a nucleic acid including more than two 5-methylcytosine residues is overrepresented in this modification and a nucleic acid with 1 or zero 5-methylcytosine residues is underrepresented. The effect of the affinity separation is to enrich for nucleic acids overrepresented in a modification in a bound phase and for nucleic acids underrepresented in a modification in an unbound phase (i.e. in solution). The nucleic acids in the bound phase can be eluted before subsequent processing.

When using MethylMiner Methylated DNA Enrichment Kit (ThermoFisher Scientific) various levels of methylation can be partitioned using sequential elutions. For example, a hypomethylated partition (e.g., no methylation) can be separated from a methylated partition by contacting the nucleic acid population with the MBD from the kit, which is attached to magnetic beads. The beads are used to separate out the methylated nucleic acids from the non-methylated nucleic acids. Subsequently, one or more elution steps are performed sequentially to elute nucleic acids having different levels of methylation. For example, a first set of methylated nucleic acids can be eluted at a salt concentration of 160 mM or higher, e.g., at least 150 mM, at least 200 mM, at least 300 mM, at least 400 mM, at least 500 mM, at least 600 mM, at least 700 mM, at least 800 mM, at least 900 mM, at least 1000 mM, or at least 2000 mM. After such methylated nucleic acids are eluted, magnetic separation is once again used to separate higher level of methylated nucleic acids from those with lower level of methylation. The elution and magnetic separation steps can repeat themselves to create various partitions such as a hypomethylated partition (representative of no methylation), a methylated partition (representative of low level of methylation), and a hyper methylated partition (representative of high level of methylation).

In some methods, nucleic acids bound to an agent used for affinity separation are subjected to a wash step. The wash step washes off nucleic acids weakly bound to the affinity agent. Such nucleic acids can be enriched in nucleic acids having the modification to an extent close to the mean or median (i.e., intermediate between nucleic acids remaining bound to the solid phase and nucleic acids not binding to the solid phase on initial contacting of the sample with the agent). The affinity separation results in at least two, and sometimes three or more partitions of nucleic acids with different extents of a modification. While the partitions are still separate, the nucleic acids of at least one partition, and usually two or three (or more) partitions are linked to nucleic acid tags, usually provided as components of adapters, with the nucleic acids in different partitions receiving different tags that distinguish members of one partition from another. The tags linked to nucleic acid molecules of the same partition can be the same or different from one another. But if different from one another, the tags may have part of their code in common so as to identify the molecules to which they are attached as being of a particular partition. For further details regarding portioning nucleic acid samples based on characteristics such as methylation, see WO2018/119452, which is incorporated herein by reference. In some embodiments, the nucleic acid molecules can be fractionated into different partitions based on the nucleic acid molecules that are bound to a specific protein or a fragment thereof and those that are not bound to that specific protein or fragment thereof.

Nucleic acid molecules can be fractionated based on DNA-protein binding. Protein-DNA complexes can be fractionated based on a specific property of a protein. Examples of such properties include various epitopes, modifications (e.g., histone methylation or acetylation) or enzymatic activity. Examples of proteins which may bind to DNA and serve as a basis for fractionation may include, but are not limited to, protein A and protein G. Any suitable method can be used to fractionate the nucleic acid molecules based on protein bound regions. Examples of methods used to fractionate nucleic acid molecules based on protein bound regions include, but are not limited to, SDS-PAGE, chromatin-immuno-precipitation (ChIP), heparin chromatography, and asymmetrical field flow fractionation (AF4).

In some embodiments, partitioning of the nucleic acids is performed by contacting the nucleic acids with a methylation binding domain (“MBD”) of a methylation binding protein (“MBP”). MBD binds to 5-methylcytosine (5mC). MBD is coupled to paramagnetic beads, such as Dynabeads® M-280 Streptavidin via a biotin linker. Partitioning into fractions with different extents of methylation can be performed by eluting fractions by increasing the NaCl concentration.

An exemplary method for molecular tag identification of MBD-bead partitioned libraries through NGS is as follows:

Physical partitioning of an extracted DNA sample (e.g., extracted blood plasma DNA from a human sample) using a methyl-binding domain protein-bead purification kit, saving all elutions from process for downstream processing.

Parallel application of differential molecular tags and NGS-enabling adapter sequences to each partition. For example, the hypermethylated, residual methylation (‘wash’), and hypomethylated partitions are ligated with NGS-adapters with molecular tags.

Re-combining all molecular tagged partitions, and subsequent amplification using adapter-specific DNA primer sequences.

Enrichment/hybridization of re-combined and amplified total library, targeting genomic regions of interest (e.g., cancer-specific genetic variants and differentially methylated regions).

Re-amplification of the enriched total DNA library, appending a sample tag. Different samples are pooled, and assayed in multiplex on an NGS instrument.

Bioinformatics analysis of NGS data, with the molecular tags being used to identify unique molecules, as well deconvolution of the sample into molecules that were differentially MBD-partitioned. This analysis can yield information on relative 5-methylcytosine for genomic regions, concurrent with standard genetic sequencing/variant detection.

Examples of MBPs contemplated herein include, but are not limited to:
(a) MeCP2 is a protein preferentially binding to 5-methyl-cytosine over unmodified cytosine.
(b) RPL26, PRP8 and the DNA mismatch repair protein MHS6 preferentially bind to 5-hydroxymethyl-cytosine over unmodified cytosine.
(c) FOXK1, FOXK2, FOXP1, FOXP4 and FOXI3 preferably bind to 5-formyl-cytosine over unmodified cytosine (Iurlaro et al., Genome Biol. 14: R119 (2013)).
(d) Antibodies specific to one or more methylated nucleotide bases.

In general, elution is a function of number of methylated sites per molecule, with molecules having more methylation eluting under increased salt concentrations. To elute the DNA into distinct populations based on the extent of methylation, one can use a series of elution buffers of increasing NaCl concentration. Salt concentration can range from about 100 nM to about 2500 mM NaCl. In one embodiment, the process results in three (3) partitions. Molecules are contacted with a solution at a first salt concentration and comprising a molecule comprising a methyl binding domain, which molecule can be attached to a capture moiety, such as streptavidin. At the first salt concentration a population of molecules will bind to the MBD and a population will remain unbound. The unbound population can be separated as a “hypomethylated” population. For example, a first partition representative of the hypomethylated form of DNA is that which remains unbound at a low salt concentration, e.g., 100 mM or 160 mM. A second partition representative of intermediate methylated DNA is eluted using an intermediate salt concentration, e.g., between 100 mM and 2000 mM concentration. This is also separated from the sample. A third partition representative of hypermethylated form of DNA is eluted using a high salt concentration, e.g., at least about 2000 mM.

The disclosure provides further methods for analyzing a population of nucleic acids in which at least some of the nucleic acids include one or more modified cytosine residues, such as 5-methylcytosine and any of the other modifications described previously. In these methods, after partitioning, the subsamples of nucleic acids are contacted with adapters including one or more cytosine residues modified at the 5C position, such as 5-methylcytosine. Preferably all cytosine residues in such adapters are also modified, or all such cytosines in a primer binding region of the adapters are modified. Adapters attach to both ends of nucleic acid molecules in the population. Preferably, the adapters include different tags of sufficient numbers that the number of combinations of tags results in a low probability e.g., 95, 99 or 99.9% of two nucleic acids with the same start and stop points receiving the same combination of tags. The primer binding sites in such adapters can be the same or different, but are preferably the same. After attachment of adapters, the nucleic acids are amplified from primers binding to the primer binding sites of the adapters. The amplified nucleic acids are split into first and second aliquots. The first aliquot is assayed for sequence data with or without further processing. The sequence data on molecules in the first aliquot is thus determined irrespective of the initial methylation state of the nucleic acid molecules. The nucleic acid molecules in the second aliquot are subjected to a procedure that affects a first nucleobase in the DNA differently from a second nucleobase in the DNA, wherein the first nucleobase comprises a cytosine modified at the 5 position, and the second nucleobase comprises unmodified cytosine. This procedure may be bisulfite treatment or another procedure that converts unmodified cytosines to uracils. The nucleic acids subjected to the procedure are then amplified with primers to the original primer binding sites of the adapters linked to nucleic acid. Only the nucleic acid molecules originally linked to adapters (as distinct from amplification products thereof) are now amplifiable because these nucleic acids retain cytosines in the primer binding sites of the adapters, whereas amplification products have lost the methylation of these cytosine residues, which have undergone conversion to uracils in the bisulfite treatment. Thus, only original molecules in the populations, at least some of which are methylated, undergo amplification. After amplification, these nucleic acids are subject to sequence analysis. Comparison of sequences determined from the first and second aliquots can indicate among other things, which cytosines in the nucleic acid population were subject to methylation.

Such an analysis can be performed using the following exemplary procedure. After partitioning, methylated DNA is linked to Y-shaped adapters at both ends including primer binding sites and tags. The cytosines in the adapters are modified at the 5 position (e.g., 5-methylated). The modification of the adapters serves to protect the primer binding sites in a subsequent conversion step (e.g., bisulfite treatment, TAP conversion, or any other conversion that does not affect the modified cytosine but affects unmodified cytosine). After attachment of adapters, the DNA molecules are amplified. The amplification product is split into two aliquots for sequencing with and without conversion. The aliquot not subjected to conversion can be subjected to sequence analysis with or without further processing. The other aliquot is subjected to a procedure that affects a first nucleobase in the DNA differently from a second nucleobase in the DNA, wherein the first nucleobase comprises a cytosine modified at the 5 position, and the second nucleobase comprises unmodified cytosine. This procedure may be bisulfite treatment or another procedure that converts unmodified cytosines to uracils. Only primer binding sites protected by modification of cytosines can support amplification when contacted with primers specific for original primer binding sites. Thus, only original molecules and not copies from the first amplification are subjected to further amplification. The further amplified molecules are then subjected to sequence analysis. Sequences can then be compared from the two aliquots. As in the separation scheme discussed above, nucleic acid tags in adapters are not used to distinguish between methylated and unmethylated DNA but to distinguish nucleic acid molecules within the same partition.

### Subjecting the First Subsample to a Procedure that Affects a First Nucleobase in the DNA Differently from a Second Nucleobase in the DNA of the First Subsample

Methods disclosed herein comprise a step of subjecting the first subsample to a procedure that affects a first nucleobase in the DNA differently from a second nucleobase in the DNA of the first subsample, wherein the first nucleobase is a modified or unmodified nucleobase, the second nucleobase is a modified or unmodified nucleobase different from the first nucleobase, and the first nucleobase and the second nucleobase have the same base pairing specificity. In some embodiments, if the first nucleobase is a modified or unmodified adenine, then the second nucleobase is a modified or unmodified adenine; if the first nucleobase is a modified or unmodified cytosine, then the second nucleobase is a modified or unmodified cytosine; if the first nucleobase is a modified or unmodified guanine, then the second nucleobase is a modified or unmodified guanine; and if the first nucleobase is a modified or unmodified thymine, then the second nucleobase is a modified or unmodified thymine (where modified and unmodified uracil are encompassed within modified thymine for the purpose of this step).

In some embodiments, the first nucleobase is a modified or unmodified cytosine, then the second nucleobase is a modified or unmodified cytosine. For example, first nucleobase may comprise unmodified cytosine (C) and the second nucleobase may comprise one or more of 5-methylcytosine (mC) and 5-hydroxymethylcytosine (hmC). Alternatively, the second nucleobase may comprise C and the first nucleobase may comprise one or more of mC and hmC. Other combinations are also possible, as indicated, e.g., in the Summary above and the following discussion, such as where one of the first and second nucleobases comprises mC and the other comprises hmC.

In some embodiments, the procedure that affects a first nucleobase in the DNA differently from a second nucleobase in the DNA of the first subsample comprises bisulfite conversion. Treatment with bisulfite converts unmodified cytosine and certain modified cytosine nucleotides (e.g. 5-formyl cytosine (fC) or 5-carboxylcytosine (caC)) to uracil whereas other modified cytosines (e.g., 5-methylcytosine, 5-hydroxylmethylcystosine) are not converted. Thus, where bisulfite conversion is used, the first nucleobase comprises one or more of unmodified cytosine, 5-formyl cytosine, 5-carboxylcytosine, or other cytosine forms affected by bisulfite, and the second nucleobase may comprise one or more of mC and hmC, such as mC and optionally hmC. Sequencing of bisulfite-treated DNA identifies positions that are read as cytosine as being mC or hmC positions. Meanwhile, positions that are read as T are identified as being T or a bisulfite-susceptible form of C, such as unmodified cytosine, 5-formyl cytosine, or 5-carboxylcytosine. Performing bisulfite conversion on a first subsample as described herein thus facilitates identifying positions containing mC or hmC using the sequence reads obtained from the first subsample. For an exemplary description of bisulfite conversion, see, e.g., Moss et al., Nat Commun. 2018; 9:5068.

In some embodiments, the procedure that affects a first nucleobase in the DNA differently from a second nucleobase in the DNA of the first subsample comprises oxidative bisulfite (Ox-BS) conversion. In some embodiments, the procedure that affects a first nucleobase in the DNA differently from a second nucleobase in the DNA of the first subsample comprises Tet-assisted bisulfite (TAB) conversion. In some embodiments, the procedure that affects a first nucleobase in the DNA differently from a second nucleobase in the DNA of the first subsample comprises Tet-assisted conversion with a substituted borane reducing agent, optionally wherein the substituted borane reducing agent is 2-picoline borane, borane pyridine, tert-butylamine borane, or ammonia borane. In some embodiments, the procedure that affects a first nucleobase in the DNA differently from a second nucleobase in the DNA of the first subsample comprises chemical-assisted conversion with a substituted borane reducing agent, optionally wherein the substituted borane reducing agent is 2-picoline borane, borane pyridine, tert-butylamine borane, or ammonia borane. In some embodiments, the procedure that affects a first nucleobase in the DNA differently from a second nucleobase in the DNA of the first subsample comprises APOBEC-coupled epigenetic (ACE) conversion.

In some embodiments, procedure that affects a first nucleobase in the DNA differently from a second nucleobase in the DNA of the first subsample comprises enzymatic conversion of the first nucleobase, e.g., as in EM-Seq. See, e.g., Vaisvila R, et al. (2019) EM-seq: Detection of DNA methylation at single base resolution from picograms of DNA. bioRxiv; DOI: 10.1101/2019.12.20.884692, available at www.biorxiv.org/content/10.1101/2019.12.20.884692v1. For example, TET2 and T4-βGT can be used to convert 5mC and 5hmC into substrates that cannot be deaminated by a deaminase (e.g., APOBEC3A), and then a deaminase (e.g., APOBEC3A) can be used to deaminate unmodified cytosines converting them to uracils.

In some embodiments, the procedure that affects a first nucleobase in the DNA differently from a second nucleobase in the DNA of the first subsample comprises separating DNA originally comprising the first nucleobase from DNA not originally comprising the first nucleobase.

In some embodiments, the first nucleobase is a modified or unmodified adenine, and the second nucleobase is a modified or unmodified adenine. In some embodiments, the modified adenine is N6-methyladenine (mA). In some embodiments, the modified adenine is one or more of N6-methyladenine (mA), N6-hydroxymethyladenine (hmA), or N6-formyladenine (fA).

Techniques comprising methylated DNA immunoprecipitation (MeDIP) can be used to separate DNA containing modified bases such as mA from other DNA. Sec, e.g., Kumar et al., Frontiers Genet. 2018; 9: 640; Greer et al., Cell 2015; 161: 868-878. An antibody specific for mA is described in Sun et al., Bioessays 2015; 37:1155-62. Antibodies for various modified nucleobases, such as forms of thymine/uracil including halogenated forms such as 5-bromouracil, are commercially available. Various modified bases can also be detected based on alterations in their base-pairing specificity. For example, hypoxanthine is a modified form of adenine that can result from deamination and is read in sequencing as a G. See, e.g., U.S. Pat. No. 8,486,630; Brown, Genomes, 2nd Ed., John Wiley & Sons, Inc., New York, N.Y., 2002, chapter 14, “Mutation, Repair, and Recombination.”

### Enriching/Capturing Step, Amplification, Adaptors, Barcodes

In some embodiments, methods disclosed herein comprise a step of capturing one or more sets of target regions of DNA, such as cfDNA. Capture may be performed using any suitable approach known in the art. In some embodiments, capturing comprises contacting the DNA to be captured with a set of target-specific probes. The set of target-specific probes may have any of the features described herein for sets of target-specific probes, including but not limited to in the embodiments set forth above and the sections relating to probes below. Capturing may be performed on one or more subsamples prepared during methods disclosed herein. In some embodiments, DNA is captured from at least the first subsample or the second subsample, e.g., at least the first subsample and the second subsample. Where the first subsample undergoes a separation step (e.g., separating DNA originally comprising the first nucleobase (e.g., hmC) from DNA not originally comprising the first nucleobase, such as hmC-seal), capturing may be performed on any, any two, or all of the DNA originally comprising the first nucleobase (e.g., hmC), the DNA not originally comprising the first nucleobase, and the second subsample. In some embodiments, the subsamples are differentially tagged (e.g., as described herein) and then pooled before undergoing capture.

The capturing step may be performed using conditions suitable for specific nucleic acid hybridization, which generally depend to some extent on features of the probes such as length, base composition, etc. Those skilled in the art will be familiar with appropriate conditions given general knowledge in the art regarding nucleic acid hybridization. In some embodiments, complexes of target-specific probes and DNA are formed.

In some embodiments, a method described herein comprises capturing cfDNA obtained from a test subject for a plurality of sets of target regions. The target regions comprise epigenetic target regions, which may show differences in methylation levels and/or fragmentation patterns depending on whether they originated from a tumor or from healthy cells. The target regions also comprise sequence-variable target regions, which may show differences in sequence depending on whether they originated from a tumor or from healthy cells. The capturing step produces a captured set of cfDNA molecules, and the cfDNA molecules corresponding to the sequence-variable target region set are captured at a greater capture yield in the captured set of cfDNA molecules than cfDNA molecules corresponding to the epigenetic target region set. For additional discussion of capturing steps, capture yields, and related aspects, sec WO2020/160414, which is incorporated herein by reference for all purposes.

In some embodiments, a method described herein comprises contacting cfDNA obtained from a test subject with a set of target-specific probes, wherein the set of target-specific probes is configured to capture cfDNA corresponding to the sequence-variable target region set at a greater capture yield than cfDNA corresponding to the epigenetic target region set.

It can be beneficial to capture cfDNA corresponding to the sequence-variable target region set at a greater capture yield than cfDNA corresponding to the epigenetic target region set because a greater depth of sequencing may be necessary to analyze the sequence-variable target regions with sufficient confidence or accuracy than may be necessary to analyze the epigenetic target regions. The volume of data needed to determine fragmentation patterns (e.g., to test for perturbation of transcription start sites or CTCF binding sites) or fragment abundance (e.g., in hypermethylated and hypomethylated partitions) is generally less than the volume of data needed to determine the presence or absence of cancer-related sequence mutations. Capturing the target region sets at different yields can facilitate sequencing the target regions to different depths of sequencing in the same sequencing run (e.g., using a pooled mixture and/or in the same sequencing cell).

In various embodiments, the methods further comprise sequencing the captured cfDNA, e.g., to different degrees of sequencing depth for the epigenetic and sequence-variable target region sets, consistent with the discussion herein. In some embodiments, complexes of target-specific probes and DNA are separated from DNA not bound to target-specific probes. For example, where target-specific probes are bound covalently or noncovalently to a solid support, a washing or aspiration step can be used to separate unbound material. Alternatively, where the complexes have chromatographic properties distinct from unbound material (e.g., where the probes comprise a ligand that binds a chromatographic resin), chromatography can be used.

As discussed in detail elsewhere herein, the set of target-specific probes may comprise a plurality of sets such as probes for a sequence-variable target region set and probes for an epigenetic target region set. In some such embodiments, the capturing step is performed with the probes for the sequence-variable target region set and the probes for the epigenetic target region set in the same vessel at the same time, e.g., the probes for the sequence-variable and epigenetic target region sets are in the same composition. This approach provides a relatively streamlined workflow. In some embodiments, the concentration of the probes for the sequence-variable target region set is greater that the concentration of the probes for the epigenetic target region set.

Alternatively, the capturing step is performed with the sequence-variable target region probe set in a first vessel and with the epigenetic target region probe set in a second vessel, or the contacting step is performed with the sequence-variable target region probe set at a first time and a first vessel and the epigenetic target region probe set at a second time before or after the first time. This approach allows for preparation of separate first and second compositions comprising captured DNA corresponding to the sequence-variable target region set and captured DNA corresponding to the epigenetic target region set. The compositions can be processed separately as desired (e.g., to fractionate based on methylation as described elsewhere herein) and recombined in appropriate proportions to provide material for further processing and analysis such as sequencing.

In some embodiments, the DNA is amplified. In some embodiments, amplification is performed before the capturing step. In some embodiments, amplification is performed after the capturing step.

In some embodiments, adapters are included in the DNA. This may be done concurrently with an amplification procedure, e.g., by providing the adapters in a 5′ portion of a primer, e.g., as described above. Alternatively, adapters can be added by other approaches, such as ligation.

In some embodiments, tags, which may be or include barcodes, are included in the DNA. Tags can facilitate identification of the origin of a nucleic acid. For example, barcodes can be used to allow the origin (e.g., subject) whence the DNA came to be identified following pooling of a plurality of samples for parallel sequencing. This may be done concurrently with an amplification procedure, e.g., by providing the barcodes in a 5′ portion of a primer, e.g., as described above. In some embodiments, adapters and tags/barcodes are provided by the same primer or primer set. For example, the barcode may be located 3′ of the adapter and 5′ of the target-hybridizing portion of the primer. Alternatively, barcodes can be added by other approaches, such as ligation, optionally together with adapters in the same ligation substrate.

Additional details regarding amplification, tags, and barcodes are discussed in the “General Features of the Methods” section below, which can be combined to the extent practicable with any of the foregoing embodiments and the embodiments set forth in the introduction and summary section.

### Captured Set

In some embodiments, a captured set of DNA (e.g., cfDNA) is provided. With respect to the disclosed methods, the captured set of DNA may be provided, e.g., by performing a capturing step after a partitioning step as described herein. The captured set may comprise DNA corresponding to a sequence-variable target region set, an epigenetic target region set, or a combination thereof. In some embodiments the quantity of captured sequence-variable target region DNA is greater than the quantity of the captured epigenetic target region DNA, when normalized for the difference in the size of the targeted regions (footprint size).

Alternatively, first and second captured sets may be provided, comprising, respectively, DNA corresponding to a sequence-variable target region set and DNA corresponding to an epigenetic target region set. The first and second captured sets may be combined to provide a combined captured set.

In some embodiments in which a captured set comprising DNA corresponding to the sequence-variable target region set and the epigenetic target region set includes a combined captured set as discussed above, the DNA corresponding to the sequence-variable target region set may be present at a greater concentration than the DNA corresponding to the epigenetic target region set, e.g., a 1.1 to 1.2-fold greater concentration, a 1.2- to 1.4-fold greater concentration, a 1.4- to 1.6-fold greater concentration, a 1.6- to 1.8-fold greater concentration, a 1.8- to 2.0-fold greater concentration, a 2.0- to 2.2-fold greater concentration, a 2.2- to 2.4-fold greater concentration a 2.4- to 2.6-fold greater concentration, a 2.6- to 2.8-fold greater concentration, a 2.8- to 3.0-fold greater concentration, a 3.0- to 3.5-fold greater concentration, a 3.5- to 4.0, a 4.0- to 4.5-fold greater concentration, a 4.5- to 5.0-fold greater concentration, a 5.0- to 5.5-fold greater concentration, a 5.5- to 6.0-fold greater concentration, a 6.0- to 6.5-fold greater concentration, a 6.5- to 7.0-fold greater, a 7.0- to 7.5-fold greater concentration, a 7.5- to 8.0-fold greater concentration, an 8.0- to 8.5-fold greater concentration, an 8.5- to 9.0-fold greater concentration, a 9.0- to 9.5-fold greater concentration, 9.5- to 10.0-fold greater concentration, a 10- to 11-fold greater concentration, an 11- to 12-fold greater concentration a 12- to 13-fold greater concentration, a 13- to 14-fold greater concentration, a 14- to 15-fold greater concentration, a 15- to 16-fold greater concentration, a 16- to 17-fold greater concentration, a 17- to 18-fold greater concentration, an 18- to 19-fold greater concentration, a 19- to 20-fold greater concentration, a 20- to 30-fold greater concentration, a 30- to 40-fold greater concentration, a 40- to 50-fold greater concentration, a 50- to 60-fold greater concentration, a 60- to 70-fold greater concentration, a 70- to 80-fold greater concentration, a 80- to 90-fold greater concentration, a 90- to 100-fold greater concentration, a 10- to 20-fold greater concentration, a 10- to 40-fold greater concentration, a 10- to 50-fold greater concentration, a 10- to 70-fold greater concentration, or a 10- to 100-fold greater concentration. The degree of difference in concentrations accounts for normalization for the footprint sizes of the target regions, as discussed in the definition section.

### Epigenetic Target Region Set

The epigenetic target region set may comprise one or more types of target regions likely to differentiate DNA from neoplastic (e.g., tumor or cancer) cells and from healthy cells, e.g., non-neoplastic circulating cells. Exemplary types of such regions are discussed in detail herein. The epigenetic target region set may also comprise one or more control regions, e.g., as described herein. In some embodiments, the epigenetic target region set has a footprint of at least 100 kb, e.g., at least 200 kb, at least 300 kb, or at least 400 kb. In some embodiments, the epigenetic target region set has a footprint in the range of 100-1000 kb, e.g., 100-200 kb, 200-300 kb, 300-400 kb, 400-500 kb, 500-600 kb, 600-700 kb, 700-800 kb, 800-900 kb, and 900-1,000 kb.

### Hypermethylation Variable Target Regions

In some embodiments, the epigenetic target region set comprises one or more hypermethylation variable target regions. In general, hypermethylation variable target regions refer to regions where an increase in the level of observed methylation, e.g., in a cfDNA sample, indicates an increased likelihood that a sample (e.g., of cfDNA) contains DNA produced by neoplastic cells, such as tumor or cancer cells. For example, hypermethylation of promoters of tumor suppressor genes has been observed repeatedly. See, e.g., Kang et al., Genome Biol. 18:53 (2017) and references cited therein. In an example, hypermethylation variable target regions can include regions that do not necessarily differ in methylation in cancerous tissue relative to DNA from healthy tissue of the same type, but do differ in methylation (e.g., have more methylation) relative to cfDNA that is typical in healthy subjects. Where, for example, the presence of a cancer results in increased cell death such as apoptosis of cells of the tissue type corresponding to the cancer, such a cancer can be detected at least in part using such hypermethylation variable target regions. In some embodiments, hypermethylation variable target regions include one or more genomic regions, where the cfDNA molecules in those regions do not differ in methylation state in cancer subjects relative to cfDNA from healthy subjects, but the presence/increased quantity of hypermethylated cfDNA in those regions is indicative of a particular tissue type (e.g., cancer origin) and is presented as cfDNA with increased apoptosis (e.g. tumor shedding) into circulation.

Hypermethylation target regions may be obtained, e.g., from the Cancer Genome Atlas. Kang et al., Genome Biology 18:53 (2017), describe construction of a probabilistic method called CancerLocator using hypermethylation target regions from breast, colon, kidney, liver, and lung. In some embodiments, the hypermethylation target regions can be specific to one or more types of cancer. Accordingly, in some embodiments, the hypermethylation target regions include one, two, three, four, or five subsets of hypermethylation target regions that collectively show hypermethylation in one, two, three, four, or five of breast, colon, kidney, liver, and lung cancers.

In some embodiments, the probes for the epigenetic target region set comprise probes specific for one or more hypermethylation variable target regions. The hypermethylation variable target regions may be any of those set forth above. For example, in some embodiments, the probes specific for hypermethylation variable target regions comprise probes specific for a plurality of loci listed in Table 1, e.g., at least 10%, 20%, 30%, 40%, 50%, 60%, 70%, 80%, 90%, or 100% of the loci listed in Table 1. In some embodiments, the probes specific for hypermethylation variable target regions comprise probes specific for a plurality of loci listed in Table 2, e.g., at least 10%, 20%, 30%, 40%, 50%, 60%, 70%, 80%, 90%, or 100% of the loci listed in Table 2. In some embodiments, the probes specific for hypermethylation variable target regions comprise probes specific for a plurality of loci listed in Table 1 or Table 2, e.g., at least 10%, 20%, 30%, 40%, 50%, 60%, 70%, 80%, 90%, or 100% of the loci listed in Table 1 or Table 2. In some embodiments, for each locus included as a target region, there may be one or more probes with a hybridization site that binds between the transcription start site and the stop codon (the last stop codon for genes that are alternatively spliced) of the gene. In some embodiments, the one or more probes bind within 300 bp of the listed position, e.g., within 200 or 100 bp. In some embodiments, a probe has a hybridization site overlapping the position listed above. In some embodiments, the probes specific for the hypermethylation target regions include probes specific for one, two, three, four, or five subsets of hypermethylation target regions that collectively show hypermethylation in one, two, three, four, or five of breast, colon, kidney, liver, and lung cancers.

### Hypomethylation Variable Target Regions

Global hypomethylation is a commonly observed phenomenon in various cancers. See, e.g., Hon et al., Genome Res. 22:246-258 (2012) (breast cancer); Ehrlich, Epigenomics 1:239-259 (2009) (review article noting observations of hypomethylation in colon, ovarian, prostate, leukemia, hepatocellular, and cervical cancers). For example, regions such as repeated elements, e.g., LINE1 elements, Alu elements, centromeric tandem repeats, pericentromeric tandem repeats, and satellite DNA, and intergenic regions that are ordinarily methylated in healthy cells may show reduced methylation in tumor cells. Accordingly, in some embodiments, the epigenetic target region set includes hypomethylation variable target regions, where a decrease in the level of observed methylation indicates an increased likelihood that a sample (e.g., of cfDNA) contains DNA produced by neoplastic cells, such as tumor or cancer cells. In an example, hypomethylation variable target regions can include regions that do not necessarily differ in methylation state in cancerous tissue relative to DNA from healthy tissue of the same type, but do differ in methylation (e.g., are less methylated) relative to cfDNA that is typical in healthy subjects. Where, for example, the presence of a cancer results in increased cell death such as apoptosis of cells of the tissue type corresponding to the cancer, such a cancer can be detected at least in part using such hypomethylation variable target regions. In some embodiments, hypomethylation variable target regions include one or more genomic regions, where the cfDNA molecules in those regions do not differ in methylation state in cancer subjects relative to cfDNA from healthy subjects, but the presence/increased quantity of hypomethylated cfDNA in those regions is indicative of a particular tissue type (e.g., cancer origin) and is presented as cfDNA with increased apoptosis (e.g. tumor shedding) into circulation.

In some embodiments, hypomethylation variable target regions include repeated elements and/or intergenic regions. In some embodiments, repeated elements include one, two, three, four, or five of LINE1 elements, Alu elements, centromeric tandem repeats, pericentromeric tandem repeats, and/or satellite DNA.

Exemplary specific genomic regions that show cancer-associated hypomethylation include nucleotides 8403565-8953708 and 151104701-151106035 of human chromosome 1. In some embodiments, the hypomethylation variable target regions overlap or comprise one or both of these regions.

In some embodiments, the probes for the epigenetic target region set comprise probes specific for one or more hypomethylation variable target regions. The hypomethylation variable target regions may be any of those set forth above. For example, the probes specific for one or more hypomethylation variable target regions may include probes for regions such as repeated elements, e.g., LINE1 elements, Alu elements, centromeric tandem repeats, pericentromeric tandem repeats, and satellite DNA, and intergenic regions that are ordinarily methylated in healthy cells may show reduced methylation in tumor cells.

In some embodiments, probes specific for hypomethylation variable target regions include probes specific for repeated elements and/or intergenic regions. In some embodiments, probes specific for repeated elements include probes specific for one, two, three, four, or five of LINE1 elements, Alu elements, centromeric tandem repeats, pericentromeric tandem repeats, and/or satellite DNA.

Exemplary probes specific for genomic regions that show cancer-associated hypomethylation include probes specific for nucleotides 8403565-8953708 and/or 151104701-151106035 of human chromosome 1. In some embodiments, the probes specific for hypomethylation variable target regions include probes specific for regions overlapping or comprising nucleotides 8403565-8953708 and/or 151104701-151106035 of human chromosome

Probes for detecting the panel of regions can include those for detecting genomic regions of interest (hotspot regions) as well as nucleosome-aware probes (e.g., KRAS codons 12 and 13) and may be designed to optimize capture based on analysis of cfDNA coverage and fragment size variation impacted by nucleosome binding patterns and GC sequence composition. Regions used herein can also include non-hotspot regions optimized based on nucleosome positions and GC models. Subjects

In some embodiments, the DNA (e.g., cfDNA) is obtained from a subject having a cancer. In some embodiments, the DNA (e.g., cfDNA) is obtained from a subject suspected of having a cancer. In some embodiments, the DNA (e.g., cfDNA) is obtained from a subject having a tumor. In some embodiments, the DNA (e.g., cfDNA) is obtained from a subject suspected of having a tumor. In some embodiments, the DNA (e.g., cfDNA) is obtained from a subject having neoplasia. In some embodiments, the DNA (e.g., cfDNA) is obtained from a subject suspected of having neoplasia. In some embodiments, the DNA (e.g., cfDNA) is obtained from a subject in remission from a tumor, cancer, or neoplasia (e.g., following chemotherapy, surgical resection, radiation, or a combination thereof). In any of the foregoing embodiments, the cancer, tumor, or neoplasia or suspected cancer, tumor, or neoplasia may be of the lung, colon, rectum, kidney, breast, prostate, or liver. In some embodiments, the cancer, tumor, or neoplasia or suspected cancer, tumor, or neoplasia is of the lung. In some embodiments, the cancer, tumor, or neoplasia or suspected cancer, tumor, or neoplasia is of the colon or rectum. In some embodiments, the cancer, tumor, or neoplasia or suspected cancer, tumor, or neoplasia is of the breast. In some embodiments, the cancer, tumor, or neoplasia or suspected cancer, tumor, or neoplasia is of the prostate. In any of the foregoing embodiments, the subject may be a human subject.

In some embodiments, the sequence-variable target region probe set has a footprint of at least 0.5 kb, e.g., at least 1 kb, at least 2 kb, at least 5 kb, at least 10 kb, at least 20 kb, at least 30 kb, or at least 40 kb. In some embodiments, the epigenetic target region probe set has a footprint in the range of 0.5-100 kb, e.g., 0.5-2 kb, 2-10 kb, 10-20 kb, 20-30 kb, 30-40 kb, 40-50 kb, 50-60 kb, 60-70 kb, 70-80 kb, 80-90 kb, and 90-100 kb.

In some embodiments, the probes specific for the sequence-variable target region set comprise probes specific for target regions from at least 10, 20, 30, or 35 cancer-related genes, such as AKT1, ALK, BRAF, CCND1, CDK2A, CTNNB1, EGFR, ERBB2, ESR1, FGFR1, FGFR2, FGFR3, FOXL2, GATA3, GNA11, GNAQ, GNAS, HRAS, IDH1, IDH2, KIT, KRAS, MED12, MET, MYC, NFE2L2, NRAS, PDGFRA, PIK3CA, PPP2R1A, PTEN, RET, STK11, TP53, and U2AF1.

### Compositions Comprising Captured DNA

Provided herein is a combination comprising first and second populations of captured DNA. The first population may comprise or be derived from DNA with a cytosine modification in a greater proportion than the second population. The first population may comprise a form of a first nucleobase originally present in the DNA with altered base pairing specificity and a second nucleobase without altered base pairing specificity, wherein the form of the first nucleobase originally present in the DNA prior to alteration of base pairing specificity is a modified or unmodified nucleobase, the second nucleobase is a modified or unmodified nucleobase different from the first nucleobase, and the form of the first nucleobase originally present in the DNA prior to alteration of base pairing specificity and the second nucleobase have the same base pairing specificity. The second population does not comprise the form of the first nucleobase originally present in the DNA with altered base pairing specificity. In some embodiments, the cytosine modification is cytosine methylation. In some embodiments, the first nucleobase is a modified or unmodified cytosine and the second nucleobase is a modified or unmodified cytosine. The first and second nucleobase may be any of those discussed herein in the Summary or with respect to subjecting the first subsample to a procedure that affects a first nucleobase in the DNA differently from a second nucleobase in the DNA of the first subsample.

In some embodiments, the first population comprises a sequence tag selected from a first set of one or more sequence tags and the second population comprises a sequence tag selected from a second set of one or more sequence tags, and the second set of sequence tags is different from the first set of sequence tags. The sequence tags may comprise barcodes.

In some embodiments, the first population comprises protected hmC, such as glucosylated hmC. In some embodiments, the first population was subjected to any of the conversion procedures discussed herein, such as bisulfite conversion, Ox-BS conversion, TAB conversion, ACE conversion, TAP conversion, TAPSβ conversion, or CAP conversion. In some embodiments, the first population was subjected to protection of hmC followed by deamination of mC and/or C. In some embodiments of the combination, the first population comprises or was derived from DNA with a cytosine modification in a greater proportion than the second population and the first population comprises first and second subpopulations, and the first nucleobase is a modified or unmodified nucleobase, the second nucleobase is a modified or unmodified nucleobase different from the first nucleobase, and the first nucleobase and the second nucleobase have the same base pairing specificity. In some embodiments, the second population does not comprise the first nucleobase. In some embodiments, the first nucleobase is a modified or unmodified cytosine, and the second nucleobase is a modified or unmodified cytosine, optionally wherein the modified cytosine is mC or hmC. In some embodiments, the first nucleobase is a modified or unmodified adenine, and the second nucleobase is a modified or unmodified adenine, optionally wherein the modified adenine is mA.

In some embodiments, the first nucleobase (e.g., a modified cytosine) is biotinylated. In some embodiments, the first nucleobase (e.g., a modified cytosine) is a product of a Huisgen cycloaddition to β-6-azide-glucosyl-5-hydroxymethylcytosine that comprises an affinity label (e.g., biotin).

In any of the combinations described herein, the captured DNA may comprise cfDNA. The captured DNA may have any of the features described herein concerning captured sets, including, e.g., a greater concentration of the DNA corresponding to the sequence-variable target region set (normalized for footprint size as discussed above) than of the DNA corresponding to the epigenetic target region set. In some embodiments, the DNA of the captured set comprises sequence tags, which may be added to the DNA as described herein. In general, the inclusion of sequence tags results in the DNA molecules differing from their naturally occurring, untagged form.

The combination may further comprise a probe set described herein or sequencing primers, each of which may differ from naturally occurring nucleic acid molecules. For example, a probe set described herein may comprise a capture moiety, and sequencing primers may comprise a non-naturally occurring label.

### Computer Systems

Methods of the present disclosure can be implemented using, or with the aid of, computer systems. For example, such methods may comprise: partitioning the sample into a plurality of subsamples, including a first subsample and a second subsample, wherein the first subsample comprises DNA with a cytosine modification in a greater proportion than the second subsample; subjecting the first subsample to a procedure that affects a first nucleobase in the DNA differently from a second nucleobase in the DNA of the first subsample, wherein the first nucleobase is a modified or unmodified nucleobase, the second nucleobase is a modified or unmodified nucleobase different from the first nucleobase, and the first nucleobase and the second nucleobase have the same base pairing specificity; and sequencing DNA in the first subsample and DNA in the second subsample in a manner that distinguishes the first nucleobase from the second nucleobase in the DNA of the first subsample.

In an aspect, the present disclosure provides a non-transitory computer-readable medium comprising computer-executable instructions which, when executed by at least one electronic processor, perform at least a portion of a method comprising: collecting cfDNA from a test subject; capturing a plurality of sets of target regions from the cfDNA, wherein the plurality of target region sets comprises a sequence-variable target region set and an epigenetic target region set, whereby a captured set of cfDNA molecules is produced; sequencing the captured cfDNA molecules, wherein the captured cfDNA molecules of the sequence-variable target region set are sequenced to a greater depth of sequencing than the captured cfDNA molecules of the epigenetic target region set; obtaining a plurality of sequence reads generated by a nucleic acid sequencer from sequencing the captured cfDNA molecules; mapping the plurality of sequence reads to one or more reference sequences to generate mapped sequence reads; and processing the mapped sequence reads corresponding to the sequence-variable target region set and to the epigenetic target region set to determine the likelihood that the subject has cancer.

The code can be pre-compiled and configured for use with a machine have a processer adapted to execute the code or can be compiled during runtime. The code can be supplied in a programming language that can be selected to enable the code to execute in a pre-compiled or as-compiled fashion.

Additional details relating to computer systems and networks, databases, and computer program products are also provided in, for example, Peterson, Computer Networks: A Systems Approach, Morgan Kaufmann, 5th Ed. (2011), Kurose, Computer Networking: A Top-Down Approach, Pearson, 7th Ed. (2016), Elmasri, Fundamentals of Database Systems, Addison Wesley, 6th Ed. (2010), Coronel, Database Systems: Design, Implementation, & Management, Cengage Learning, 11th Ed. (2014), Tucker, Programming Languages, McGraw-Hill Science/Engineering/Math, 2nd Ed. (2006), and Rhoton, Cloud Computing Architected: Solution Design Handbook, Recursive Press (2011), each of which is hereby incorporated by reference in its entirety.

### Cancer and Other Diseases

The present methods can be used to diagnose presence of conditions, particularly cancer, in a subject, to characterize conditions (e.g., staging cancer or determining heterogeneity of a cancer), monitor response to treatment of a condition, effect prognosis risk of developing a condition or subsequent course of a condition. The present disclosure can also be useful in determining the efficacy of a particular treatment option. Successful treatment options may increase the amount of copy number variation or rare mutations detected in subject's blood if the treatment is successful as more cancers may die and shed DNA. In other examples, this may not occur. In another example, perhaps certain treatment options may be correlated with genetic profiles of cancers over time. This correlation may be useful in selecting a therapy.

Additionally, if a cancer is observed to be in remission after treatment, the present methods can be used to monitor residual disease or recurrence of disease.

In some embodiments, the methods and systems disclosed herein may be used to identify customized or targeted therapies to treat a given disease or condition in patients based on the classification of a nucleic acid variant as being of somatic or germline origin. Typically, the disease under consideration is a type of cancer. Non-limiting examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic leukemia (CLL), chronic myeloid leukemia (CML), chronic myelomonocytic leukemia (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas. Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma. Type and/or stage of cancer can be detected from genetic variations including mutations, rare mutations, indels, copy number variations, transversions, translocations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine.

Genetic data can also be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

Further, the methods of the disclosure may be used to characterize the heterogeneity of an abnormal condition in a subject. Such methods can include, e.g., generating a genetic profile of extracellular polynucleotides derived from the subject, wherein the genetic profile comprises a plurality of data resulting from copy number variation and rare mutation analyses. In some embodiments, an abnormal condition is cancer. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease. Again, in the example of cancer, there may be multiple tumor foci, perhaps where one or more foci are the result of metastases that have spread from a primary site.

The present methods can be used to generate or profile, fingerprint or set of data that is a summation of genetic information derived from different cells in a heterogeneous disease. This set of data may comprise copy number variation, epigenetic variation, and mutation analyses alone or in combination.

The present methods can be used to diagnose, prognose, monitor or observe cancers, or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

Non-limiting examples of other genetic-based diseases, disorders, or conditions that are optionally evaluated using the methods and systems disclosed herein include achondroplasia, alpha-1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolemia, familial Mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (SCID), sickle cell disease, spinal muscular atrophy, Tay-Sachs, thalassemia, trimethylaminuria, Turner syndrome, velocardiofacial syndrome, WAGR syndrome, Wilson disease, or the like.

In some embodiments, a method described herein comprises detecting a presence or absence of DNA originating or derived from a tumor cell at a preselected timepoint following a previous cancer treatment of a subject previously diagnosed with cancer using a set of sequence information obtained as described herein. The method may further comprise determining a cancer recurrence score that is indicative of the presence or absence of the DNA originating or derived from the tumor cell for the test subject. Where a cancer recurrence score is determined, it may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

In some embodiments, a cancer recurrence score is compared with a predetermined cancer recurrence threshold, and the test subject is classified as a candidate for a subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy.

The methods discussed above may further comprise any compatible feature or features set forth elsewhere herein, including in the section regarding methods of determining a risk of cancer recurrence in a test subject and/or classifying a test subject as being a candidate for a subsequent cancer treatment.

### Methods of Determining a Risk of Cancer Recurrence in a Test Subject and/or Classifying a Test Subject as Being a Candidate for a Subsequent Cancer Treatment.

In some embodiments, a method provided herein is a method of determining a risk of cancer recurrence in a test subject. In some embodiments, a method provided herein is a method of classifying a test subject as being a candidate for a subsequent cancer treatment.

Any of such methods may comprise collecting DNA (e.g., originating or derived from a tumor cell) from the test subject diagnosed with the cancer at one or more preselected timepoints following one or more previous cancer treatments to the test subject. The subject may be any of the subjects described herein. The DNA may be cfDNA. The DNA may be obtained from a tissue sample.

Any of such methods may comprise capturing a plurality of sets of target regions from DNA from the subject, wherein the plurality of target region sets comprises a sequence-variable target region set and an epigenetic target region set, whereby a captured set of DNA molecules is produced. The capturing step may be performed according to any of the embodiments described elsewhere herein. In any of such methods, the previous cancer treatment may comprise surgery, administration of a therapeutic composition, and/or chemotherapy.

Any of such methods may comprise sequencing the captured DNA molecules, whereby a set of sequence information is produced. The captured DNA molecules of the sequence-variable target region set may be sequenced to a greater depth of sequencing than the captured DNA molecules of the epigenetic target region set.

Any of such methods may comprise detecting a presence or absence of DNA originating or derived from a tumor cell at a preselected timepoint using the set of sequence information. The detection of the presence or absence of DNA originating or derived from a tumor cell may be performed according to any of the embodiments thereof described elsewhere herein.

Methods of determining a risk of cancer recurrence in a test subject may comprise determining a cancer recurrence score that is indicative of the presence or absence, or amount, of the DNA originating or derived from the tumor cell for the test subject. The cancer recurrence score may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

Methods of classifying a test subject as being a candidate for a subsequent cancer treatment may comprise comparing the cancer recurrence score of the test subject with a predetermined cancer recurrence threshold, thereby classifying the test subject as a candidate for the subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy. In some embodiments, the subsequent cancer treatment comprises chemotherapy or administration of a therapeutic composition.

Any of such methods may comprise determining a disease-free survival (DFS) period for the test subject based on the cancer recurrence score; for example, the DFS period may be 1 year, 2 years, 3, years, 4 years, 5 years, or 10 years.

In some embodiments, the set of sequence information comprises sequence-variable target region sequences, and determining the cancer recurrence score may comprise determining at least a first subscore indicative of the amount of SNVs, insertions/deletions, CNVs and/or fusions present in sequence-variable target region sequences.

In some embodiments, a number of mutations in the sequence-variable target regions chosen from 1, 2, 3, 4, or 5 is sufficient for the first subscore to result in a cancer recurrence score classified as positive for cancer recurrence. In some embodiments, the number of mutations is chosen from 1, 2, or 3.

In some embodiments, the set of sequence information comprises epigenetic target region sequences, and determining the cancer recurrence score comprises determining a second subscore indicative of the amount of molecules (obtained from the epigenetic target region sequences) that represent an epigenetic state different from DNA found in a corresponding sample from a healthy subject (e.g., cfDNA found in a blood sample from a healthy subject, or DNA found in a tissue sample from a healthy subject where the tissue sample is of the same type of tissue as was obtained from the test subject). These abnormal molecules (i.e., molecules with an epigenetic state different from DNA found in a corresponding sample from a healthy subject) may be consistent with epigenetic changes associated with cancer, e.g., methylation of hypermethylation variable target regions and/or perturbed fragmentation of fragmentation variable target regions, where “perturbed” means different from DNA found in a corresponding sample from a healthy subject.

In some embodiments, a proportion of molecules corresponding to the hypermethylation variable target region set and/or fragmentation variable target region set that indicate hypermethylation in the hypermethylation variable target region set and/or abnormal fragmentation in the fragmentation variable target region set greater than or equal to a value in the range of 0.001%-10% is sufficient for the second subscore to be classified as positive for cancer recurrence. The range may be 0.001%-1%, 0.005%-1%, 0.01%-5%, 0.01%-2%, or 0.01%-1%.

In some embodiments, any of such methods may comprise determining a fraction of tumor DNA from the fraction of molecules in the set of sequence information that indicate one or more features indicative of origination from a tumor cell. This may be done for molecules corresponding to some or all of the epigenetic target regions, e.g., including one or both of hypermethylation variable target regions and fragmentation variable target regions (hypermethylation of a hypermethylation variable target region and/or abnormal fragmentation of a fragmentation variable target region may be considered indicative of origination from a tumor cell). This may be done for molecules corresponding to sequence variable target regions, e.g., molecules comprising alterations consistent with cancer, such as SNVs, indels, CNVs, and/or fusions. The fraction of tumor DNA may be determined based on a combination of molecules corresponding to epigenetic target regions and molecules corresponding to sequence variable target regions.

Determination of a cancer recurrence score may be based at least in part on the fraction of tumor DNA, wherein a fraction of tumor DNA greater than a threshold in the range of 10-11 to 1 or 10-10 to 1 is sufficient for the cancer recurrence score to be classified as positive for cancer recurrence. In some embodiments, a fraction of tumor DNA greater than or equal to a threshold in the range of 10-10 to 10-9, 10-9 to 10-8, 10-8 to 10-7, 10-7 to 10-6, 10-6 to 10-5, 10-5 to 10-4, 10-4 to 10-3, 10-3 to 10-2, or 10-2 to 10-1 is sufficient for the cancer recurrence score to be classified as positive for cancer recurrence. In some embodiments, the fraction of tumor DNA greater than a threshold of at least 10-7 is sufficient for the cancer recurrence score to be classified as positive for cancer recurrence. A determination that a fraction of tumor DNA is greater than a threshold, such as a threshold corresponding to any of the foregoing embodiments, may be made based on a cumulative probability. For example, the sample was considered positive if the cumulative probability that the tumor fraction was greater than a threshold in any of the foregoing ranges exceeds a probability threshold of at least 0.5, 0.75, 0.9, 0.95, 0.98, 0.99, 0.995, or 0.999. In some embodiments, the probability threshold is at least 0.95, such as 0.99.

In some embodiments, the set of sequence information comprises sequence-variable target region sequences and epigenetic target region sequences, and determining the cancer recurrence score comprises determining a first subscore indicative of the amount of SNVs, insertions/deletions, CNVs and/or fusions present in sequence-variable target region sequences and a second subscore indicative of the amount of abnormal molecules in epigenetic target region sequences, and combining the first and second subscores to provide the cancer recurrence score. Where the first and second subscores are combined, they may be combined by applying a threshold to each subscore independently (e.g., greater than a predetermined number of mutations (e.g., >1) in sequence-variable target regions, and greater than a predetermined fraction of abnormal molecules (i.e., molecules with an epigenetic state different from the DNA found in a corresponding sample from a healthy subject; e.g., tumor) in epigenetic target regions), or training a machine learning classifier to determine status based on a plurality of positive and negative training samples.

In some embodiments, a value for the combined score in the range of −4 to 2 or −3 to 1 is sufficient for the cancer recurrence score to be classified as positive for cancer recurrence.

In any embodiment where a cancer recurrence score is classified as positive for cancer recurrence, the cancer recurrence status of the subject may be at risk for cancer recurrence and/or the subject may be classified as a candidate for a subsequent cancer treatment.

In some embodiments, the cancer is any one of the types of cancer described elsewhere herein, e.g., colorectal cancer.

### Therapies and Related Administration

In certain embodiments, the methods disclosed herein relate to identifying and administering customized therapies to patients given the status of a nucleic acid variant as being of somatic or germline origin. In some embodiments, essentially any cancer therapy (e.g., surgical therapy, radiation therapy, chemotherapy, and/or the like) may be included as part of these methods. Typically, customized therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In certain embodiments, the status of a nucleic acid variant from a sample from a subject as being of somatic or germline origin may be compared with a database of comparator results from a reference population to identify customized or targeted therapies for that subject. Typically, the reference population includes patients with the same cancer or disease type as the test subject and/or patients who are receiving, or who have received, the same therapy as the test subject. A customized or targeted therapy (or therapies) may be identified when the nucleic variant and the comparator results satisfy certain classification criteria (e.g., are a substantial or an approximate match).

In certain embodiments, the customized therapies described herein are typically administered parenterally (e.g., intravenously or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by methods such as, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the disclosure described herein may be employed in practicing the invention. It is therefore contemplated that the disclosure shall also cover any such alternatives, modifications, variations or equivalents. It is intended that the following claims define the scope of the invention and that methods and structures within the scope of these claims and their equivalents be covered thereby.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, computer readable media, and/or component features, steps, elements, or other aspects thereof can be used in various combinations.

### Cancer Treatments, Therapies

In some cases, the cancer treatment includes, without limitation, imatinib, gefatinib, afatinib, dacomitinib, sunitinib, sorafenib, vandetanib, brivanib, cabozantib, neratinib, tivantinib, bevacizumab, cixutumumab, dalotuzumab, figitumumab, rilotumumab, onartuzumab, ganitumab, ramucirumab, ridaforolimus, tensirolimus, everolimus, BMS-690514, BMS-754807, EMD 525797, GDC-0973, GDC-0941, MK-2206, AZD6244, GSK1120212, PX-866, XL821, IMC-A12, MM-121, PF-02341066, RG7160, and Sym004. Antibodies suitable for use as anti-EGFR therapy include cetuximab (Trade Name: Erbitux) and panitumumab (Trade Name: Vectibex). In some cases. In some cases, the cancer treatment includes EGFR tyrosine kinase inhibitors such as gefitinib (Trade Name: Iressa), erlotinib (Trade Name: Tarceva), lapatinib, canertinib, and cetuximab.

In some instances, therapties may be used in combination, such as an anti-EGFR therapy and an anti-EGFR therapy. Anti-EGFR therapy may be used in combination with any combination of chemotherapeutic agents or chemotherapeutic regimens, for example, FOLFOX (fluorouracil [5-FU]/leucovorin/oxaliplatin), FOLFIRI (5-FU/leucovorin/irinotecan), and the like.

In some aspects, a cancer treatment is administered to a subject. In some cases, the cancer treatment is administered in combination another therapy, such as a non-anti-EGFR therapy with anti-EGFR therapy.

### Genetic Analysis

Genetic analysis includes detection of nucleotide sequence variants and copy number variations. Genetic variants can be determined by sequencing. The sequencing method can be massively parallel sequencing, that is, simultaneously (or in rapid succession) sequencing any of at least 100,000, 1 million, 10 million, 100 million, or 1 billion polynucleotide molecules. Sequencing methods may include, but are not limited to: high-throughput sequencing, pyrosequencing, sequencing-by-synthesis, single-molecule sequencing, nanopore sequencing, semiconductor sequencing, sequencing-by-ligation, sequencing-by-hybridization, RNA-Seq (Illumina), Digital Gene Expression (Helicos), Next-generation sequencing, Single Molecule Sequencing by Synthesis (SMSS) (Helicos), massively-parallel sequencing, Clonal Single Molecule Array (Solexa), shotgun sequencing, Maxam-Gilbert or Sanger sequencing, primer walking, sequencing using PacBio, SOLiD, Ion Torrent, or Nanopore platforms and any other sequencing methods known in the art.

Sequencing can be made more efficient by performing sequence capture, that is, the enrichment of a sample for target sequences of interest, e.g., sequences including the KRAS and/or EGFR genes or portions of them containing sequence variant biomarkers. Sequence capture can be performed using immobilized probes that hybridize to the targets of interest.

Cell free DNA can include small amounts of tumor DNA mixed with germline DNA. Sequencing methods that increase sensitivity and specificity of detecting tumor DNA, and, in particular, genetic sequence variants and copy number variation, can be useful in the methods of this invention. Such methods are described in, for example, in WO 2014/039556. These methods not only can detect molecules with a sensitivity of up to or greater than 0.1%, but also can distinguish these signals from noise typical in current sequencing methods. Increases in sensitivity and specificity from blood-based samples of cfDNA can be achieved using various methods. One method includes high efficiency tagging of DNA molecules in the sample, e.g., tagging at least any of 50%, 75% or 90% of the polynucleotides in a sample. This increases the likelihood that a low-abundance target molecule in a sample will be tagged and subsequently sequenced, and significantly increases sensitivity of detection of target molecules.

Another method involves molecular tracking, which identifies sequence reads that have been redundantly generated from an original parent molecule, and assigns the most likely identity of a base at each locus or position in the parent molecule. This significantly increases specificity of detection by reducing noise generated by amplification and sequencing errors, which reduces frequency of false positives.

Methods of the present disclosure can be used to detect genetic variation in non-uniquely tagged initial starting genetic material (e.g., rare DNA) at a concentration that is less than 5%, 1%, 0.5%, 0.1%, 0.05%, or 0.01%, at a specificity of at least 99%, 99.9%, 99.99%, 99.999%, 99.9999%, or 99.99999%. Sequence reads of tagged polynucleotides can be subsequently tracked to generate consensus sequences for polynucleotides with an error rate of no more than 2%, 1%, 0.1%, or 0.01%.

Copy number variation determination can involve determining a quantitative measure of polynucleotides in a sample mapping to a genetic locus, such as the EGFR gene or KRAS gene. The quantitative measure can be a number. Once the total number of polynucleotides mapping to a locus is determined, this number can be used in standard methods of determining Copy Number Variation at the locus. A quantitative measure can be normalized against a standard. In one method, a quantitative measure at a test locus can be standardized against a quantitative measure of polynucleotides mapping to a control locus in the genome, such as gene of known copy number. In another method, the quantitative measure can be compared against the amount of nucleic acid in the original sample. For example, the quantitative measure can be compared against an expected measure for diploidy. In another method, the quantitative measure can be normalized against a measure from a control sample, and normalized measures at different loci can be compared. In another method, quantifying involves quantifying parent or original molecules in a sample mapping to a locus, rather than number of sequence reads. A copy number variation may be an amplification or a deletion or truncation of a gene. An amplification may be 3, 4, 5, 6, 7, 8, 9, 10, or 10 or more copies of a gene. A deletion or truncation may be 0 or 1 copies of a gene.

An example of a method for detecting copy number variation may include an array. The array may comprise a plurality of capture probes. The capture probes can be oligonucleotides that are bound to the surface of the array. The capture probes may bind to at least 1, 2, 3, 4, 5, 6, 7, 8, 9, 10, 11, or 12 genes as set forth in Table 1. DNA derived from the subject may be labeled (e.g., with a fluorophore) prior to hybridization for detection.

In other examples, a gene of interest may be amplified using primers that recognize the gene of interest. The primers may hybridize to a gene upstream and/or downstream of a particular region of interest (e.g., upstream of a mutation site). A detection probe may be hybridized to the amplification product. Detection probes may specifically hybridize to a wild-type sequence or to a mutated/variant sequence. Detection probes may be labeled with a detectable label (e.g., with a fluorophore). Detection of a wild-type or mutant sequence may be performed by detecting the detectable label (e.g., fluorescence imaging). In examples of copy number variation, a gene of interest may be compared with a reference gene. Differences in copy number between the gene of interest and the reference gene may indicate amplification or deletion/truncation of a gene. Examples of platforms suitable to perform the methods described herein include digital PCR platforms such as e.g., Fluidigm Digital Array.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

Treatment efficacy as involving promoter methylation: In one example, of interest is understanding whether gedatolisib will sensitize advanced TNBC or BRCA1/2 mutant breast cancers to PARP inhibition with talazoparib. In a phase I study, 3 patients were classified as partial response (PR), and 5 patients as stable disease (SD). Tissue genomics were used and could not account for confounding scenarios of patients who progress vs. those who do not. Importantly, an alternative mechanism of gene inactivation exists which can account for the difference via promoter methylation.

PI3K inhibitors thought to reduce nuclear pools (potentially leading to replication errors/fork stalling/increased DNA repair) This would increase repair mechanisms and reliance on PARP. PI3K inhibitors also thought to impede PI3K interaction with HR complex. This would increase reliance on PARP for DNA repair

Here, BRCA promoter methylation status for these patients is evaluated by analyzing TNBC samples and clinical outcomes data, which includes addition of promoter methylation data to compute an HRD score.

### Example 2

MLH1 promoter methylation testing: In another example, one can identify patients at risk for genetic/familial forms of colorectal cancer or Lynch syndrome-associated tumor types. MLH1 promoter hypermethylation (and often, BRAF V600E positive) is associated with sporadic forms of CRC.

### Example 3

BRCA1 promoter methylation testing: In another example, one can include as a variant type for homologous recombination repair deficiency-associated tumor types (brca, ovca, panc, prca). Many HRD related tumors exhibit single copy loss or rearrangement of BRCA1, without a second hit. A portion of these cases are likely to possess promoter hypermethylation of the remaining allele, leading to biallelic loss of BRCA1.

### Example 4

MGMT promoter methylation testing: In another example, one can incorporate promoter hypermethylation associated with benefit when treated with certain types of chemotherapy.

### Example 5

Allelic Expression and promoter methylation: The aforementioned techniques for quantifying levels of promoter methylation allow for determination of methylation levels in an allelic specific manner.

Using the methods and composition described herein, the methylation level of the one or more classification regions to characterize the sample, including determination of a quantitative measure. Determination of a quantitative measure can include combining a plurality of nucleic acids derived from at least one of blood or tissue of a subject with a solution including an amount of methyl binding domain (MBD) proteins to produce a nucleic acid-MBD protein solution; and performing a plurality of washes of the nucleic acid-MBD protein solution with a salt solution to produce a number of nucleic acid fractions. In some instances, individual nucleic acid fractions having a threshold number of methylated cytosines in regions of the plurality of nucleic acids having at least the threshold cytosine-guanine content. Thereafter, a wash of the plurality of washes is performed with a solution having a concentration of sodium chloride (NaCl) and produces a nucleic acid fraction of the number of nucleic acid fractions having a range of binding strengths to MBD proteins.

One may determine that a first nucleic acid fraction is associated with a first partition of a plurality of partitions of nucleic acids, the first partition corresponding to a first range of binding strengths to MBD proteins; attach a first molecular barcode to nucleic acids of the first nucleic acid fraction, the first molecular barcode being included in a first set of molecular barcodes associated with the first partition, and subsequently determine that a second nucleic acid fraction is associated with a second partition of the plurality of partitions of nucleic acids, the second partition corresponding to a second range of binding energies to MBD proteins different from the first range of binding strengths to MBD proteins; and thereafter attach a second molecular barcode to nucleic acids of the second nucleic acid fraction, the second molecular barcode being included in a second set of molecular barcodes associated with the second partition.

Using the aforementioned methods and compositions, one can determine the ratio of the number of molecules that overlap a classification region normalized by total positive control molecules, wherein the molecules exhibit a threshold amount of methylated cytosines. In some instances, this quantitative measure is compared to a predetermined threshold value to call methylation status of the one or more classification regions. Also, in some instances, determining the ratio comprises filtering of a molecule based at least on a threshold amount of methylated cytosines and/or determining a methylation level of the one or more classification regions is based on the number of methylated CpGs.

Canonically, the importance of specific DNA methylation patterns for developmentally appropriate gene expression is most clearly demonstrated for imprinted loci. Whereas genes are normally expressed from both the maternal and the paternal alleles, at an imprinted loci, only the maternal or the paternal allele is expressed. In some instances, this restriction may be limited to specific tissues or times during development.

The methylation status of the DNA surrounding an imprinted locus also displays a pattern that is unique to each allele. The locations of the differentially methylated domains or regions (DMDs or DMRs) are variable and the expressed allele may show both hypo- and/or hypermethylated domains. Parental allele-specific methylation patterns may direct allele-specific expression. In the context of cancer, one example is the H19/Igf2 and Rasgrf1 loci and their DMRs have enhancer blocking activity and bind CTCF in a methylation-sensitive manner. CTCF bound to an unmethylated DMR represses enhancer to promoter interactions needed for Igf2 and Rasgrf1 expression and this block is relieved allowing expression when the DMRs are methylated and CTCF binding is prevented.

### Example 6

Promoter methylation silencing, imprinting: Conventional understanding is that imprinted genes are “silenced”, which this form of mono-allelic expression originating from either the maternal or paternal allele. In cancers, some silenced imprinting genes' copies could be reactivated, leading to expressions from both alleles. The loss of monoallelic gene regulation is named loss of imprinting (LOI), and in addition, amplifications of the activated copies of imprinted genes without affecting the methylation of the silenced copy have also been observed in cancer cell lines [19]. In such instances, the imprinted genes could be expressed in two or more transcription sites instead of one. Therefore, the increased number of transcription site detections of imprinted genes in the cell nuclei could be used as potential cancer biomarkers. Here, existing reports detect nascent RNA or pre-mRNA in situ hybridization (ISH) method to target introns can be used to visualize and label these transcription sites with applications to study the transcriptional regulations of both imprinted genes.

Using the methods and composition described herein, the methylation level of the one or more classification regions to characterize the sample, including determination of a quantitative measure including determining the ratio of the number of molecules that overlap a classification region normalized by total positive control molecules, wherein the molecules exhibit a threshold amount of methylated cytosines. In some instances, this quantitative measure is compared to a predetermined threshold value to call methylation status of the one or more classification regions. Also, in some instances, determining the ratio comprises filtering of a molecule based at least on a threshold amount of methylated cytosines and/or determining a methylation level of the one or more classification regions is based on the number of methylated CpGs.

### Example 7

Other forms of expression resulting from epigenetic allelic status, imprinting: As described, conventional understanding is that imprinted genes leading to LOI make cells susceptible to cellular transformation and tumourigenesis through aberrant biallelic expression (e.g., imprinted IGF2 locus is thought to promote tumourigenesis by inhibiting apoptosis in colorectal cancer and to lead to over-proliferation defects in lung, colon and ovarian cancer and LOI of other imprinted genes, such as H19, PEG3, MEST and PLAGL1, in varying cancers).

Nevertheless, whereas LOI is associated with silencing of the normally active allele major expression through downregulation of reportedly imprinted genes in cancer is less understood. For example, in oesophageal cancer, LOI of IGF2 was specifically associated with expression downregulation, and improved survival. Also in prostate cancer, no increased expression was found for IGF2 despite LOI. Notwithstanding the major relevance of LOI in cancer, this fragmentary evidence demonstrates that the current paradigm of the role of LOI in cancer (i.e. growth & tumour promoting expression) requires additional evaluation.

The aforementioned methods allow determination of imprinted gene networks in which these genes are co-regulated. In parallel, copy number variation (CNV) can be an important cause of imprinting deregulation in cancer, with multi-modal detection of genomic and epigenomic features provided by the aforementioned methods and techniques.

The aforementioned methods and techniques support systematic analyses of LOI, or other forms of allelic expression that are still lacking. Whereas monoallelic expression is better understood, only few regions are well-characterised in humans, without understanding of evaluated tissue-specific imprinting patterns given the existing methods using to detect aberrant monoallelic expression on cancer at single imprinted loci. Moreover, the practical applicability of existing high-throughput methods is greatly hampered by the necessity for genotyping. The aforementioned techniques allow for the systematical profiling of (i) allelic expression including monoallelically expressed/imprinted loci and (ii) their dysregulation and deregulation (e.g., LOI) in cancer.

### Example 8

Imbalance of epigenetic regulation can also increase the plasticity of tumor cells, epigenetic allelic expression for determination of tumor heterogeneity: Given the apparent importance epigenetic regulation, including allelic expression and its role in cancer pathogenesis, of interest is applying the aforementioned methods and compositions in the context of ascertaining tumor heterogeneity. Various cancers (e.g., breast cancer) are highly complex heterogeneous disease at the molecular level forming tumor subpopulations with distinct phenotypic characteristics. Differences in DNA methylation pattern between different cell subpopulations can drive phenotypic changes, which is valuable for providing novel insights into the intratumor epigenetic heterogeneity of breast cancer. In some instances, manual observation of epiallelic expression has been utilized to identify differential epialleles between tumor core and tumor periphery and characterized tumor subpopulations with distinct methylation patterns. A method of epiallelic imbalance can be calculated on the basis of Jensen-Shannon divergence, although it is readily understood by one of skill that a variety of other methods for calculating variation can be utilized. This technique can identifying continuous CpGs (e.g., four continuous CpGs covered by the same read as an epiallele. Given the methylation status of a CpG was methylated or unmethylated, an epiallele contained 16 possible methylation patterns. Divergence (e.g., entropic divergence) can be utilized to quantify the dissimilarity between methylation patterns of one or more samples.

Here, methylation patterns of tumor (e.g., core tumor) are likely more disordered than tumor periphery as a result of higher epigenetic heterogeneity and consequently genes with higher epigenetic heterogeneity also had higher transcriptional heterogeneity. Using the aforementioned methods and techniques, this can be systematically analyzed to evaluate the panoply of epigenetic states within the tumor. We defined four continuous CpGs covered by the same read as an epiallele.

### Example 9

Measuring epigenetic promoter methylation divergence, epiallele diversity for shifting and epigenetic burden: In other instances, loci with epigenetic allelic variance can be calculated using compositional entropy equation (e.g., Methyclone). Here, the epigenetic state of each locus as involving cytosine methylation at four consecutive CpG dinucleotides support a possible 16 CpG methylation patterns at these loci as an epiallele. Epigenetic shifts in loci can be regarded as significant when the epiallele proportions at these sites undergo a statistically significant entropy shift (calculated by delta Boltzmann entropy ΔS<Δ90) in their composition when comparing one or more samples.

Determination of epigenetic status per million loci can be applied to normalize the variable depth of coverage per specimen and the number of loci measured, to determine the overall magnitude of epiallele shifting across the genome as a form of calculating epiallele burden, analogous to tumor mutation burden. Epigenetic shifting can include both gain and/or loss of epialleles between two specimens. The epiallele and systematic epigenetic loci measurements can be determined using methylome data from the aforementioned methods and compositions.

In various embodiments, (e.g., em-SEQ, ERRBS) can be used to validate a subset of specimens using orthogonal methylome sequencing methods. This systematic analysis allows determination of tumor genetic and epigenetic heterogeneity to characterize independent, biologically distinct phenomena, each presumably with a unique functional significance. The degree of epigenetic allelic burden can and can not include other factors, such as age and other clinical parameter, somatic mutations affecting epigenetic modifier genes (e.g., DNMT3A, TET2, and IDH1/2), behavior of dominant epigenetic alleles in a similar or distinct manner from genetic alleles during clonal evolution, also at serial timepoints, between the kinetics and pattern of genetic and epigenetic alleles during progression when longitudinally monitored.

In various instances, these measurements can be utilized to classified, including through use of a machine learning algorithm (e.g., vector support machine) and/or various databases, to identify one or more of epiallele pattern kinetics and somatic mutation burdens during disease progression. For example, diagnostic criteria may be divided into disease with predominant epiallele diversity and low somatic mutations (e.g., epigenetically-driven) and others with lower epiallele diversity and higher mutation burden (e.g., genetically-driven). The latter develop increasing epigenetic diversity upon progression. Here, in both cases, genetic clonal composition remains predominantly stable, although instances of genetic clonal stability are also likely to be identified. In the absence of a link between epigenetic instability and genetic instability or specific somatic mutations alternative modes of dominant heterogeneity in diagnosed patients can be evaluated as involvin: one genetic and one epigenetic form of dominance.

### Example 10

Measurements for epigenetic shifts, allelic expression: Here, the ability to determine system wide biological gene activation/inactivation dependent on epigenetic allelic status, and associated clinical cancer risk, including methylation status of epialleles, quantification of methylation events that span over several CpGs, can be regarded as a form of haplotype definition (e.g., accounting for both methylation status of individual CpGs within the sequence read as well as the average methylation level of the sequence read itself).

Using next generation sequencing (NGS)-based data with exemplary default values (minimum 2 CpG sites, minimum average methylation beta value of 0.5 for CpG), an optional thresholding measurement defines a subpopulation of epialleles of interest and is based on the minimum number and the average methylation level of cytosines in various sequence contexts (e.g., CpG, CHG, or CHH). The thresholding parameters can be fully adjustable to target desired population of epialleles; sites, maximum average methylation beta value of 0.1 for non-CpG sites). Optional thresholding of sequence reads without thresholding, includes methylation beta value for every genomic location is computed as a ratio of a number of methylated cytosines to total number of methylated and unmethylated cytosines: b=C/(C+T). In contrast, when read thresholding is performed (default mode of action), the level of methylation per every genomic position, a Variant Epiallele Frequency (VEF), can be calculated as a ratio of a number of methylated cytosines in read pairs passing the threshold (Ca) to total number of methylated and unmethylated cytosines in all read pairs: VEF=Ca/(C+T).

Adjusting scope to include a level of extended genomic regions rather than individual bases, VEF can be equal to the ratio of a number of read pairs passing threshold (Na) to the total number of read pairs (N) overlapping the region of interest: VEF=Na/N. This allows a group of epialleles (i.e., individual methylation patterns) with similar methylation properties to be defined by thresholding, wherein VEF effectively represents the frequency of this group of epialleles passing the threshold at the level of individual cytosines or extended genomic regions.

In either case, methylation beta values as well as VEF values from default reporting mode with read thresholding can be produced from any number of BAM files without prior hypothesis, if the experimental setup allows to call methylation on per-base level. Both of these values effectively represent methylation levels per genomic position and, as such, can be directly used further as an input for other bioinformatic tools including, but not limited to, differential methylation analysis tools.

1. A method, comprising:
detecting methylation in one or more promoter regions of at least one of a plurality of genes; and
generating a plurality of methylation calls to quantify methylation of the one or more promoter regions.

2. The method of claim 1, comprising obtaining a sample.

3. The method of claim 1, comprising having obtained a sample.

4. The method of claim 1, comprising processing the quantities of methylation of the one or more promoter regions to characterize a sample.

5. The method of claim 1, wherein characterizing the sample comprises homologous recombination deficiency (HRD), cancer derived promoter methylation, familial forms of colorectal cancer, or Lynch syndrome tumor types.

6. The method of claim 1, wherein the promoter comprises a region of 5kb upstream of the transcription start site (TSS), wherein the 5kb region is further refined using one or more of: costume panel regions, methylation peaks found in clinical samples, and excluding peaks found in normal samples.

7. The method of claim 6, wherein the TSS is defined at the transcript level.

8. The method of claim 6, wherein the TSS is defined at the gene level.

9. The method of claim 1, comprising determining the ratio of the number of molecules that overlap a target region normalized by total positive control molecules.

10. The method of claim 9, wherein determining the ratio comprises filtering of a molecule based at least on the number of overlapping CpGs.

11. The method of claim 1, wherein the quantifying of methylation of the one or more promoter regions is based on the number of methylated CpGs.

12. The method of claim 1, comprising refining the one or more promoter regions based at least on literature annotations, common methylation peak positions, and/or public datasets.

13. The method of claim 1, wherein the genes comprise tumor suppressor genes, homologous recombination deficiency (HRR) genes, and immuno-oncology (IO) genes.

14. The method of claim 13, wherein the HRR genes comprise at least BRCA1 and BRCA2.

15. The method of claim 1, comprising comparing to a minimum methylation threshold derived from a population of training samples.

16. The method of claim 15, wherein the training samples comprise cancer-free samples.

17. The method of claim 15, wherein the minimum methylation threshold for calling comprises at least one of: a minimum molecule count of 1-100 and a minimum methylation score per gene is the max of: 95 quantile in normal+8×10⁵ or Median+5*median absolute deviation.

18. The method of claim 1, wherein quantifying methylation of the one or more promoter regions is predictive of therapy response.

19. The method of claim 18, wherein quantifying methylation of the one or more promoter regions is combined with an microsatellite instability-high (MSI-H) status.

20. The method of claim 18, wherein the therapy comprises one or more of an immune checkpoint inhibitor, poly (ADP-ribose) polymerase (PARP) inhibitor, a kinase inhibitor, or an aromatase inhibitor, or a PI3K and mTOR inhibitor.

21. The method of claim 20, wherein the immune checkpoint inhibitor is Pembrolizumab.

22. The method of claim 20, wherein the poly (ADP-ribose) polymerase (PARP) inhibitor Olaparib or Talazoparib.

23. The method of claim 20, wherein the therapy is a combination of a PI3K and mTOR inhibitor and a poly (ADP-ribose) polymerase (PARP) inhibitor.

24. The method of claim 23, wherein the PI3K and mTOR inhibitor is Gedatolisib and the poly (ADP-ribose) polymerase (PARP) inhibitor is Talazoparib.

25. A method comprising:
determining promoter regions of at least one of a plurality of genes, each obtained from a plurality of samples;
determining methylation scores for the promoter regions to generate a plurality of methylation calls and/or quantification of promoter methylation;
processing the plurality of methylation calls to generate a prediction that a test sample exhibits a genomic state.

26. A method, comprising:
obtaining, by a computing system having one or more hardware processors and memory, sequencing reads derived from a sample of a subject,
determining one or more classification regions corresponding to a plurality of genes included in the sample; and
determine a methylation level of the one or more classification regions by generating a quantitative measure derived from the sequencing reads in the sample of the subject.

27-49. (canceled)
